SEQUENCE LISTING (24 sequences)

SEQ ID NO: 1 - RNA, 29 nt, synthetic construct
  source 1..29: mol_type = other RNA
ggagaacaactckaggcagaggtctcaaa

SEQ ID NO: 2 - RNA, 29 nt, synthetic construct
  source 1..29: mol_type = other RNA
tttgagacctctgtccngagttgttctcc

SEQ ID NO: 3 - RNA, 29 nt, synthetic construct
  source 1..29: mol_type = other RNA
  misc_structure 13: note = DNA
tttgagacctctgtccrgagttgttctcc

SEQ ID NO: 4 - RNA, 29 nt, synthetic construct
  source 1..29: mol_type = other RNA
  modified_base 13: mod_base = OTHER; note = 3-deaza-2'-deoxyadenosine
tttgagacctctgtccngagttgttctcc

SEQ ID NO: 5 - RNA, 29 nt, synthetic construct
  source 1..29: mol_type = other RNA
ccggcaggaagcgaaaagctgaggccgac

SEQ ID NO: 6 - RNA, 29 nt, synthetic construct
  source 1..29: mol_type = other RNA
gtcggcctcagctttcsgcttcctgccgg

SEQ ID NO: 7 - RNA, 32 nt, synthetic construct
  source 1..32: mol_type = other RNA
  modified_base 13: mod_base = OTHER; note = 8-azanebularine
gctcgcgatgcgngagggctctgatagctacg

SEQ ID NO: 8 - RNA, 32 nt, synthetic construct
  source 1..32: mol_type = other RNA
cgtagctatcagagccccccggcatcgcgagc

SEQ ID NO: 9 - RNA, 29 nt, synthetic construct
  source 1..29: mol_type = other RNA
ccggcaggaagtgaaaagctgaggccgac

SEQ ID NO: 10 - RNA, 29 nt, synthetic construct
  source 1..29: mol_type = other RNA
gtcggcctcagctttcrgcttcctgccgg

SEQ ID NO: 11 - RNA, 29 nt, synthetic construct
  source 1..29: mol_type = other RNA
  misc_structure 17: note = DNA
gtcggcctcagctttcrgcttcctgccgg

SEQ ID NO: 12 - RNA, 29 nt, synthetic construct
  source 1..29: mol_type = other RNA
  modified_base 17: mod_base = OTHER; note = 7-deaza-2'-deoxyguanosine
gtcggcctcagctttcngcttcctgccgg

SEQ ID NO: 13 - RNA, 29 nt, synthetic construct
  source 1..29: mol_type = other RNA
  modified_base 17: mod_base = OTHER; note = 8-bromo-2'-deoxyguanosine
gtcggcctcagctttcngcttcctgccgg

SEQ ID NO: 14 - RNA, 29 nt, synthetic construct
  source 1..29: mol_type = other RNA
  modified_base 17: mod_base = OTHER; note = 7-deaza-2'-deoxyadenosine
gtcggcctcagctttcngcttcctgccgg

SEQ ID NO: 15 - RNA, 29 nt, synthetic construct
  source 1..29: mol_type = other RNA
  modified_base 17: mod_base = OTHER; note = 2'-ara-fluoro adenosine
gtcggcctcagctttcngcttcctgccgg

SEQ ID NO: 16 - RNA, 29 nt, synthetic construct
  source 1..29: mol_type = other RNA
  modified_base 17: mod_base = OTHER; note = 2'-ara-fluoro guanosine
gtcggcctcagctttcngcttcctgccgg

SEQ ID NO: 17 - RNA, 29 nt, synthetic construct
  source 1..29: mol_type = other RNA
  modified_base 17: mod_base = OTHER; note = 2'-fluoroadenosine
gtcggcctcagctttcngcttcctgccgg

SEQ ID NO: 18 - RNA, 29 nt, synthetic construct
  source 1..29: mol_type = other RNA
  modified_base 17: mod_base = OTHER; note = 2'-fluoroguanosine
gtcggcctcagctttcngcttcctgccgg

SEQ ID NO: 19 - RNA, 29 nt, synthetic construct
  source 1..29: mol_type = other RNA
  modified_base 17: mod_base = OTHER; note = 2'-fluoroinosine
gtcggcctcagctttcngcttcctgccgg

SEQ ID NO: 20 - RNA, 29 nt, synthetic construct
  source 1..29: mol_type = other RNA
  modified_base 17: mod_base = i; note = DNA
gtcggcctcagctttcngcttcctgccgg

SEQ ID NO: 21 - RNA, 29 nt, synthetic construct
  source 1..29: mol_type = other RNA
  modified_base 17: mod_base = i
gtcggcctcagctttcngcttcctgccgg

SEQ ID NO: 22 - RNA, 29 nt, synthetic construct
  source 1..29: mol_type = other RNA
  modified_base 17: mod_base = OTHER; note = 8-aza-inosine
gtcggcctcagctttcngcttcctgccgg

SEQ ID NO: 23 - RNA, 29 nt, synthetic construct
  source 1..29: mol_type = other RNA
  modified_base 17: mod_base = OTHER; note = 1'-(2-amino-8-methyl-4-oxo-1,4-dihydro-6-quinazolin yl)-2'-deoxyribose
gtcggcctcagctttcngcttcctgccgg

SEQ ID NO: 24 - RNA, 29 nt, synthetic construct
  source 1..29: mol_type = other RNA
  modified_base 17: mod_base = OTHER; note = 3-deaza-2'-deoxyadenosine
gtcggcctcagctttcngcttcctgccgg

(19) United States
(12) Patent Application Publication
(10) Pub. No.: US 2026/0258410 A1
(43) Pub. Date: Sep. 3, 2026

(54) OLIGONUCLEOTIDES FOR ADAR-MEDIATED RNA EDITING AND USE THEREOF

(71) Applicants: ProQR Therapeutics II B.V., Leiden (NL); The Regents of the University of California, Oakland, CA (US)

(72) Inventors: Peter BEAL, Oakland, CA (US); Erin E. DOHERTY, Oakland, CA (US); Agya KARKI, Oakland, CA (US); Allison WILCOX, Oakland, CA (US); Andrew FISHER, Oakland, CA (US); Peter Christian DE VISSER, Leiden (NL)

(73) Assignees: ProQR Therapeutics II B.V., Leiden (NL); The Regents of the University of California, Oakland, CA (US)

(21) Appl. No.: 18/994,572

(22) PCT Filed: Jul. 14, 2023
(86) PCT No.: PCT/EP2023/069612

§ 371 Date: Jan. 14, 2025

Related U.S. Application Data
(60) Provisional application No. 63/389,441, filed on Jul. 15, 2022.
(60) Provisional application No. 63/481,566, filed on Jan. 25, 2023.

(51) Int. Cl. C12N 15/113; A61K 31/7115; A61K 31/7125; C12N 9/78; C12N 15/11; C12Q 1/6869

(52) U.S. Cl. CPC C12N 15/113; A61K 31/7115; A61K 31/7125; C12N 9/78; C12N 15/111; C12Q 1/6869; C12Y 305/04004; C12N 2310/11; C12N 2310/31; C12N 2310/33

(57) ABSTRACT

The invention relates to antisense oligonucleotides that can form a double stranded nucleic acid complex with a target RNA molecule, wherein the double stranded nucleic acid complex is capable of recruiting an adenosine deaminating enzyme for deamination of a target adenosine in the target RNA molecule, wherein the nucleotide directly 5′ of the target adenosine in the target RNA molecule is a guanosine (5′-G), and wherein the nucleotide in the AON that is opposite the guanosine is a nucleotide analog that can induce a syn conformation of the guanosine.

### TECHNICAL FIELD

The invention relates to the field of medicine, and more specifically to the field of RNA editing whereby an RNA molecule in a cell is targeted by an antisense oligonucleotide (AON) to change the chemical properties of a specific nucleotide within the target RNA molecule. The invention relates to RNA-editing AONs (also referred to as ‘EONs’) that comprise at least one nucleotide analog to improve their in vivo and in vitro RNA editing effect.

### BACKGROUND

RNA editing is a natural process through which eukaryotic cells alter the sequence of their RNA molecules, often in a site-specific and precise way, thereby increasing the repertoire of genome encoded RNAs by several orders of magnitude. RNA editing enzymes have been described for eukaryotic species throughout the animal and plant kingdoms, and these processes play an important role in managing cellular homeostasis in metazoans from the simplest life forms (such as Caenorhabditis elegans) to humans. Examples of RNA editing are adenosine (A) to inosine (I) conversions and cytidine (C) to uridine (U) conversions, which occur through enzymes called Adenosine Deaminases acting on RNA (ADAR) and APOBEC/AID (cytidine deaminases that act on RNA), respectively.

ADAR is a multi-domain protein, comprising a catalytic domain, and two to three double-stranded RNA recognition domains, depending on the enzyme in question. Each recognition domain recognizes a specific double stranded RNA (dsRNA) sequence and/or conformation. The catalytic domain does also play a role in recognizing and binding a part of the dsRNA helix, although the key function of the catalytic domain is to convert an A into I in a nearby, more or less predefined, position in the target RNA, by deamination of the nucleobase. Inosine is read as guanosine by the translational machinery of the cell, meaning that, if an edited adenosine is in a coding region of an mRNA or pre-mRNA, it can recode the protein sequence. A to I conversions may also occur in 5′ non-coding sequences of a target mRNA, creating new translational start sites upstream of the original start site, which gives rise to N-terminally extended proteins, or in the 3′ UTR or other non-coding parts of the transcript, which may affect the processing and/or stability of the RNA. In addition, A to I conversions may take place in splice elements in introns or exons in pre-mRNAs, thereby altering the pattern of splicing. As a result, exons may be included or skipped. The enzymes catalysing adenosine deamination are within an enzyme family of ADARs, which include human deaminases hADAR1 and hADAR2, as well as hADAR3. However, for hADAR3 no deaminase activity has been shown yet.

The use of oligonucleotides to edit a target RNA applying adenosine deaminase has been described (e.g., Woolf et al. 1995. PNAS 92:8298-8302; Montiel-Gonzalez et al. PNAS 2013, 110(45):18285-18290; Vogel et al. 2014. Angewandte Chemie Int Ed 53:267-271). A disadvantage of the method described by Montiel-Gonzalez et al. (2013) is the need for a fusion protein consisting of the boxB recognition domain of bacteriophage lambda N-protein, genetically fused to the adenosine deaminase domain of a truncated natural ADAR protein. It requires target cells to be either transduced with the fusion protein, which is a major hurdle, or that target cells are transfected with a nucleic acid construct encoding the engineered adenosine deaminase fusion protein for expression. The system described by Vogel et al. (2014) suffers from similar drawbacks, in that it is not clear how to apply the system without having to genetically modify the ADAR first and subsequently transfect or transform the cells harboring the target RNA, to provide the cells with this genetically engineered protein. U.S. Pat. No. 9,650,627 describes a similar system. The oligonucleotides of Woolf et al. (1995) that were 100% complementary to the target RNA sequences suffered from severe lack of specificity: nearly all adenosines in the target RNA strand that was complementary to the antisense oligonucleotide were edited.

It is known in the art that ADAR may act on any dsRNA. Through a process sometimes referred to as ‘promiscuous editing’, the enzyme will edit multiple A's in the dsRNA. Hence, there is a need for methods and means that circumvent such promiscuous editing and only target specific adenosines in a target RNA molecule to become therapeutic applicable. Vogel et al. (2014) showed that such off-target editing can be suppressed by using 2′-O-Me-modified nucleosides in the oligonucleotide at positions opposite to adenosines that should not be edited and used a non-modified nucleoside directly opposite to the specifically targeted adenosine on the target RNA. However, the specific editing effect at the target nucleotide has not been shown to take place without the use of recombinant ADAR enzymes having covalent bonds with the AON. Several publications have now shown that the recruitment of endogenous ADAR (hence without the need for an exogenous and/or recombinant source) is feasible while maintaining a specificity in which a single adenosine within a target RNA molecule can be targeted and deaminated to an inosine. WO2016/097212 discloses antisense oligonucleotides (AONs) for the targeted editing of RNA, wherein the AONs are characterized by a sequence that is complementary to a target RNA sequence (therein referred to as the ‘targeting portion’) and by the presence of a stem-loop structure (therein referred to as the ‘recruitment portion’), which is preferably non-complementary to the target RNA. Such oligonucleotides are referred to as ‘self-looping AONs’. The recruitment portion acts in recruiting a natural ADAR enzyme present in the cell to the dsRNA formed by hybridization of the target sequence with the targeting portion. Due to the recruitment portion, there is no need for conjugated entities or presence of modified recombinant ADAR enzymes. WO2016/097212 describes the recruitment portion as being a stem-loop structure mimicking either a natural substrate (e.g., the GluB receptor) or a Z-DNA structure known to be recognized by the dsRNA binding domains, or Z-DNA binding domains, of ADAR enzymes. A stem-loop structure can be an intermolecular stem-loop structure, formed by two separate nucleic acid strands, or an intramolecular stem loop structure, formed within a single nucleic acid strand. The stem-loop structure of the recruitment portion as described is an intramolecular stem-loop structure, formed within the AON itself, and able to attract (endogenous) ADAR. Similar stem-loop structure-comprising systems have been described in WO2017/050306, WO2020/001793, WO2017/010556, WO2020/246560, and WO2022/078995.

WO2017/220751 and WO2018/041973 describe a next generation kind of AONs that do not comprise such a stem-loop structure but that are (almost fully) complementary to the targeted area, except for one or more mismatching nucleotides, ‘wobbles’, or ‘bulges’. The sole mismatch may be at the site of the nucleoside opposite the target adenosine, but in other embodiments AONs were described with multiple bulges and/or wobbles when attached to the target sequence area. It appeared possible to achieve in vitro, ex vivo and in vivo RNA editing with AONs lacking a stem-loop structure and with endogenous ADAR enzymes when the sequence of the AON was carefully selected such that it could attract ADAR. The ‘orphan nucleoside’, which is defined as the nucleoside in the AON that is positioned directly opposite the target adenosine in the target RNA molecule, did not carry a 2′-O-Me modification. The orphan nucleoside could also be a deoxyribonucleoside (DNA, carrying no 2′ modification in the sugar entity), wherein the remainder of the AON did carry 2′-O-alkyl modifications at the sugar entity (such as 2′-O-Me), or the nucleotides directly surrounding the orphan nucleoside contained particular chemical modifications (such as DNA in comparison to RNA) that further improved the RNA editing efficiency and/or increased the resistance against nucleases. Such effects could even be further improved by using sense oligonucleotides (SONs) that ‘protected’ the AONs against breakdown (described in WO2018/134301). The use of chemical modifications and particular structures in oligonucleotides that could be used in ADAR-mediated editing of specific adenosines in a target RNA have been the subject of numerous publications in the field, such as WO2019/111957, WO2019/158475, WO2020/165077, WO2020/201406, WO2020/211780, WO2021/008447, WO2021/020550, WO2021/060527, WO2021/117729, WO2021/136408, WO2021/182474, WO2021/216853, WO2021/242778, WO2021/242870, WO2021/242889, WO2022/007803, WO2022/018207, WO2022/026928, and WO2022/124345. The use of specific sugar moieties has been disclosed in for instance WO2020/154342, WO2020/154343, WO2020/154344, WO2022/103839, and WO2022/103852, whereas the use of stereo-defined linker moieties (in general for oligonucleotides that for instance can be used for exon skipping, in gapmers, in siRNA, or specifically for RNA-editing oligonucleotides, related to a wide variety of target sequences) has been disclosed in WO2011/005761, WO2014/010250, WO2014/012081, WO2015/107425, WO2017/015575 (HTT), WO2017/062862, WO2017/160741, WO2017/192664, WO2017/192679 (DMD), WO2017/198775, WO2017/210647, WO2018/067973, WO2018/098264, WO2018/223056 (PNPLA3), WO2018/223073 (APOC3), WO2018/223081 (PNPLA3), WO2018/237194, WO2019/032607 (C9orf72), WO2019/055951, WO2019/075357 (SMA/ALS), WO2019/200185 (DM1), WO2019/217784 (DM1), WO2019/219581, WO2020/118246 (DM1), WO2020/160336 (HTT), WO2020/191252, WO2020/196662, WO2020/219981 (USH2A), WO2020/219983 (RHO), WO2020/227691 (C9orf72), WO2021/071788 (C9orf72), WO2021/071858, WO2021/178237 (MAPT), WO2021/234459, WO2021/237223, and WO2022/099159.

Next to these disclosures, an extensive number of publications relate to the targeting of specific RNA target molecules, or specific adenosines within such RNA target molecules, be it to repair a mutation that resulted in a premature stop codon, or some kind of other mutation causing disease. Examples of such disclosures in which adenosines are targeted within specified target RNA molecules are WO2020/157008 and WO2021/136404 (USH2A); WO2021/113270 (APP); WO2021/113390 (CMT1A); WO2021/209010 (IDUA, Hurler syndrome); WO2021/231673 and WO2021/242903 (LRRK2); WO2021/231675 (ASS1); WO2021/231679 (GJB2); WO2019/071274 and WO2021/231680 (MECP2); WO2021/231685 and WO2021/231692 (OTOF, autosomal recessive non-syndromic hearing loss); WO2021/231691 (XLRS); WO2021/231698 (argininosuccinate lyase deficiency); WO2021/130313 and WO2021/231830 (ABCA4); and WO2021/243023 (SERPINA1).

Despite the numerous and wide variety of achievements outlined above, there remains a need for improved compounds that can utilise (endogenous) cellular pathways and enzymes that have deaminase activity, such as naturally expressed ADAR enzymes to edit specifically and more efficiently endogenous nucleic acids in mammalian cells, even in whole organisms, to alleviate disease.

### SUMMARY OF THE INVENTION

The invention relates to an antisense oligonucleotide (AON) capable of forming a double stranded nucleic acid complex with a target RNA molecule, wherein the double stranded nucleic acid complex is capable of recruiting an adenosine deaminating enzyme for deamination of a target adenosine in the target RNA molecule, wherein the nucleotide directly 5′ of the target adenosine in the target RNA molecule is a guanosine, and wherein the nucleotide in the AON that is opposite the guanosine is a nucleotide analog that can induce a syn conformation of the guanosine. In a preferred embodiment, the nucleotide analog comprises a modified nucleobase, more preferably wherein the nucleotide analog comprises a modified purine nucleobase, even more preferably wherein the modified purine nucleobase comprises a 3-deaza-purine modification, optionally a 3-deaza-adenine modification. In one embodiment, the modified purine nucleobase comprises a 7-deaza-adenine modification. In one embodiment, the modified purine nucleobase comprises a hydrogen bond donor at N1 that has a pKa that is higher than 3.7 and lower than 9.5.

In one embodiment, the invention relates to an AON according to the invention, wherein the nucleotide analog is selected from the group consisting of:
7-deaza-2′-deoxyadenosine (7-deaza dA);
7-deaza-2′-adenosine (7-deaza A);
7-deaza-2′-deoxy-2′-fluoroadenosine (7-deaza fA);
7-deaza-2′-deoxy-2′-ara-fluoroadenosine;
7-deaza-2′-deoxy-2′,2′-difluoroadenosine;
3-deaza-2′-deoxyadenosine (3-deaza dA);
3-deaza-2′-adenosine (3-deaza A);
3-deaza-2′-deoxy-2′-fluoroadenosine (3-deaza fA);
3-deaza-2′-deoxy-2′-ara-fluoroadenosine;
3-deaza-2′-deoxy-2′,2′-difluoroadenosine;
3,7-dideaza-2′-deoxyadenosine (3,7-dideaza dA);
3,7-dideaza-2′-adenosine (3,7-dideaza A);
3,7-dideaza-2′-deoxy-2′-fluoroadenosine (3,7-dideaza fA);
3,7-dideaza-2′-deoxy-2′-ara-fluoroadenosine;
3,7-dideaza-2′-deoxy-2′,2′-difluoroadenosine;
3-deaza-2′-O-[2-(methoxy)ethyl]adenosine;
3-deaza-2′-O-[2-methylamino-2-oxoethyl]adenosine;
2′-deoxy-2′-fluoroguanosine;
2′-ara-fluoro guanosine (FANA G);
2′,2′-difluoro guanosine;
2′-deoxyinosine (dl);
2′-OH-inosine (rl);
2′-fluoroinosine (2′-F-1);
2′-ara-fluoro inosine (FANA I);
2′,2′-difluoro inosine;
5-formylindole-2′-deoxyriboside;
5-formyl-2′-fluoro-2′-deoxyriboside
5-formylindole-2′-ara-fluoro-2′-deoxyriboside;
5-formylindole-2′,2′-difluoro-2′-deoxyriboside;
5-formylindole-2′-O-methylriboside;
5-formylindole-2′-O-[2-(methoxy)ethyl]riboside;
5-formylindole-2′-O-[2-methylamino-2-oxoethyl]riboside
beta-(4-amidino-1H-imidazol-1-yl) riboside;
beta-(4-amidino-1H-imidazol-1-yl) 2′-deoxyriboside;
beta-(4-amidino-1H-imidazol-1-yl) 2′-ara-fluoro-2′-deoxyriboside; and beta-(4-amidino-1H-imidazol-1-yl) 2′,2′-difluoro-2′-deoxyriboside.

In a preferred embodiment, the adenosine deaminating enzyme is an endogenous ADAR enzyme, preferably ADAR1 or ADAR2.

The invention further relates to a pharmaceutical composition comprising an AON according to the invention, and a pharmaceutically acceptable carrier or diluent. The invention also relates to an AON according to the invention, or a pharmaceutical composition according to the invention, for use in the treatment, amelioration, or slowing down progression of a genetic disease caused by a premature UGA termination codon.

The invention also relates to a method for the deamination of at least one target adenosine present in a target RNA molecule in a cell, wherein the nucleotide 5′ of the target adenosine in the RNA molecule is a guanosine, the method comprising the steps of: (i) providing the cell with an AON according to the invention, or a pharmaceutical composition according to the invention; (ii) allowing annealing of the AON to the target RNA molecule to form a double stranded nucleic acid complex capable of recruiting an adenosine deaminating enzyme in the cell, preferably an endogenous adenosine deaminating enzyme; (iii) allowing the adenosine deaminating enzyme to deaminate the target adenosine in the target RNA molecule; and (iv) optionally identifying the presence of the deaminated adenosine in the target RNA molecule. In a further preferred embodiment, step (iv) comprises: (a) sequencing a region of the target RNA molecule, wherein the region comprises the deaminated target adenosine; (b) assessing the presence of a functional, elongated, full length and/or wild type protein when the target adenosine is in a UGA stop codon; or (c) using a functional read-out, wherein the target RNA molecule after the deamination encodes a functional, full length, elongated and/or wild type protein.

The invention also relates to a method for the deamination of at least one target adenosine present in a target RNA molecule, the method comprising the steps of: (i) providing an AON according to the invention, or a pharmaceutical composition according to the invention; (ii) allowing annealing of the AON to the target RNA molecule to form a double stranded nucleic acid complex with the target RNA molecule; (iii) allowing a mammalian adenosine deaminating enzyme to deaminate the target adenosine in the target RNA molecule; and (iv) optionally identifying the presence of the deaminated adenosine in the target RNA molecule. Preferably, the adenosine deaminating enzyme is an endogenous ADAR enzyme, preferably ADAR1 or ADAR2.

### BRIEF DESCRIPTION OF THE DRAWINGS

One or more embodiments of the invention will now be described, by way of example only, with reference to the accompanying drawings, in which:

FIGS. 1A-1F show in vitro deamination kinetics for ADAR2 and ADAR1 p110 varying 5′ nearest neighbor base pairing. (FIG. 1A) Sequence of model substrate for ADARs with varying base pairing adjacent to the editing site (X:Y). The target sequence (upper strand; SEQ ID NO:1) is from the human IDUA transcript. The lower strand is the AON (SEQ ID NO:2-4). Y is the 5′ nucleotide from the target A in the target sequence and is either G or U. X is the 3′ nucleotide from the orphan C (here deoxycytidine (dC)), and is either U, A, G, or C. (FIG. 1B) Comparison of rate constants for reaction with 100 nM ADAR2, showing the best results when G (Y) pairs with G (X). (FIG. 1C) Comparison of rate constants with 250 nM ADAR1 p110, showing the best results when G (Y) pairs with A or G (X). (FIG. 1D) Comparison of rate constants with 10 nM ADAR2, showing that even though proper editing takes place when G (Y) pairs with G (X), it is preferred that the 5′ nucleotide in the target sequence Y is U and pairs with A (X). See Table 1 for fitted values. (FIG. 1E) Duplex substrates where target sequence (upper strand; SEQ ID NO:5) is derived from wild-type human MECP2 transcript in which the 5′-G adjacent to the editing site A is a G and pairs with either C or G in the lower strand which represents that AON (SEQ ID NO:6). (FIG. 1F) Comparison of rate constants for reaction with 100 nM ADAR2. Clearly, the best results are obtained when the 5′-G pairs with G. See Table 2 for fitted values.

FIGS. 2A-2C show the characterization of a complex formed between ADAR2 RD E488Q and a 32 bp 8-azanebularine (N) containing duplex with G:G pair (32 bp GG RNA) adjacent to N, which represents the target nucleotide. (FIG. 2A) (Top) Sequence of 32 bp duplex used for crystallization. The upper strand is SEQ ID NO:7, and the lower strand representing an AON is SEQ ID NO:8. (Bottom) EMSA gel of hADAR2-RD E488Q with this duplex. Protein concentrations were as follows: lane 1: no protein, lanes 2-12: 0.25, 0.5, 1, 2, 4, 8, 16, 32, 64, 128, 256 nM. (FIG. 2B) Quantification of protein binding by EMSA. (FIG. 2C) Fit of a G_{syn}:Gₐₙₜᵢ base pair in the electron density map. “5′ G” indicates guanosine linked to N on its 5′ side. “−1 G” refers to the guanosine in the opposite strand on the 3′ side of the orphan base paired with the 5′-G.

FIGS. 3A-3B show that the G_{syn}:Gₐₙₜᵢ pair accommodates G489 in the minor groove. The 2-amino group of the 5′-G in the target RNA relative to G489 is given by an arrow. (FIG. 3A) (Left) Space filling representation of the G_{syn}:Gₐₙₜᵢ pair and location of the 2-amino group relative to G489 in the complex. (Middle) Chemical structure of the G_{syn}:Gₐₙₜᵢ pair. (Right) Chemical structure of a Gₐₙₜᵢ:Gₐₙₜᵢ pair and the clash that the 2-amino group would have with G489 in this conformation. (FIG. 3B) Space filling representation of U:A pair and its location relative to G489 in a complex with ADAR2 for the ideal 5′ nearest neighbor base pair (Matthews et al. Nat Struct Mol Biol 2016. 23(5):426-433).

FIGS. 4A-4C show the in vitro deamination kinetics for ADAR2 and duplex RNAs with nucleoside analogs paired with the 5′-G. (FIG. 4A) Sequence of model substrate for ADAR2 derived from the R255X mutant of the human MECP2 mRNA varying base pairing to the 5′ G adjacent to the editing site (X). The upper strand is SEQ ID NO:9 and the lower strand is SEQ ID NO:10-24. X=guanosine (G), adenosine (A), 2′-deoxyguanosine (dG), 2′-deoxyadenosine (dA), 7-deaza-2′-deoxyguanosine (7-deaza dG), 8-bromo-2′-deoxyguanosine (8-bromo dG) and 3-deaza-2′-deoxyadenosine (3-deaza dA). (FIG. 4B) Comparison of rate constants for reaction with 100 nM ADAR2. See Table 3 for fitted values. (FIG. 4C) The G_{syn}:AH+ₐₙₜᵢ pair (Pan B. et al. Biochemistry, 1999. 38:2826-2831).

FIGS. 5A-5B show the in vitro deamination kinetics for ADAR1 p110 and duplex RNAs with nucleoside analogs paired with the 5′-G. (FIG. 5A) Sequence of model RNA substrate derived from the human IDUA mRNA and the guide oligonucleotide are as shown in FIG. 1A. X=2′-deoxyguanosine (dG), 2′-deoxyadenosine (dA) and 3-deaza-2′-deoxyadenosine (3-deaza dA) (FIG. 5B) Comparison of rate constants for reaction with 250 nM ADAR1 p110. See Table 4 for fitted values.

FIG. 6 shows a set of additional nucleotide analogs that were tested for in vitro deamination kinetics for ADAR2 and duplex RNAs and that pair with the 5′-G using a model substrate for ADAR2 derived from the R255X mutant of the human MECP2 mRNA identical to the sequence shown in FIG. 4A. FANA A is a 2′-Fluoro-Arabino adenosine, FANA G is a 2′-Fluoro-Arabino guanosine, 7-deaza-dA is a 7-deaza-2′-deoxyadenosine, 2′F A is a 2′-fluoro adenosine, 2′F G is a 2′-fluoro guanosine, 2′F I is a 2′-fluoro inosine, dl is a 2′-deoxyinosine, rl is a 2-OH inosine, 8-aza I is a 8-aza-2′-OH inosine, and yC is a monomer according to formula (1):

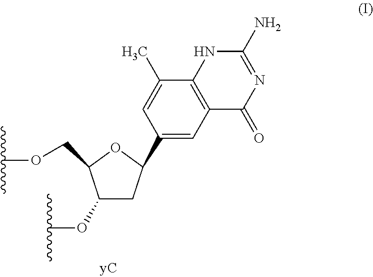 [SMILES: *OC[C@H]1O[C@@H](c2cc(C)c3[nH]c(N)nc(=O)c3c2)C[C@@H]1O*]

FIG. 7 shows the rate constant for ADAR2-catalyzed adenosine deamination at the target site using the 10 modified nucleic acids shown in FIG. 6, in comparison to the 3-deaza dA nucleotide analog.

### DETAILED DESCRIPTION

There is a constant need for improving the pharmacokinetic properties of RNA-editing antisense oligonucleotides (AONs, sometimes referred to as ‘editing oligonucleotides’, or ‘EONs’) without negatively affecting editing efficiency of the target adenosine in the target RNA. Many chemical modifications exist and may be applied in the generation of AONs, whose properties are not always compatible with the desire of achieving efficient RNA editing.

Mutagenesis studies of human ADAR2 revealed that a single mutation at residue 488 from glutamate to glutamine (E488Q), gave an increase in the rate constant of deamination by 60-fold when compared to the wild-type enzyme (Kuttan and Bass. Proc Natl Acad Sci USA 2012. 109(48):3295-3304). During the deamination reaction, ADAR flips the edited base out of its RNA duplex, and into the enzyme active site (Matthews et al. 2016). When ADAR2 edits adenosines in the preferred context (an A:C mismatch) the nucleotide opposite the target adenosine is often referred to as the ‘orphan cytidine’. The crystal structure of ADAR2 E488Q bound to double stranded RNA (dsRNA) revealed that the glutamine (Gln) side chain at position 488 can donate an H-bond to the N3 position of the orphan cytidine, which leads to the increased catalytic rate of ADAR2 E488Q. In the wild-type enzyme, wherein a glutamate (Glu) is present at position 488 instead of a glutamine (Gln) the amide group of the glutamine is absent and is instead a carboxylic acid. To obtain the same contact of the orphan cytidine with the E488Q mutant would then, for the wild-type situation, require protonation for this contact to occur. To make use of endogenously expressed ADAR2 to correct disease relevant mutations, it is essential to maximize the editing efficiency of the wild type ADAR2 enzyme present in the cell. WO2020/252376 discloses the use of AONs with modified RNA bases, especially at the position of the orphan cytidine to mimic the hydrogen-bonding pattern observed by the E488Q ADAR2 mutant. By replacing the nucleotide opposite the target adenosine in the AON with cytidine analogs that serve as H-bond donors at N3, it was envisioned that it would be possible to stabilize the same contact that is believed to provide the increase in catalytic rate for the mutant enzyme. Two cytidine analogs were of particular interest: pseudoisocytidine (also referred to as ‘piC’; Lu et al. J Org Chem 2009. 74(21):8021-8030; Burchenal et al. (1976) Cancer Res 36:1520-1523) and Benner's base Z (also referred to as ‘dZ’; Yang et al. Nucl Acid Res 2006. 34(21):6095-6101) that were initially selected because they offer hydrogen-bond donation at N3 with minimal perturbation to the shape of the nucleobase. The presence of the cytidine analog in the AON may exist in addition to modifications to the ribose 2′ group. The ribose 2′ groups in the AON can be independently selected from 2′-H (i.e., DNA), 2′-OH (i.e., RNA), 2′-O-Me, 2′-MOE, 2′-F, or 2′-4′-linked (i.e., a bridged nucleic acid such as a locked nucleic acid (LNA)), or other 2′ substitutions. The 2′-4′ linkage can be selected from linkers known in the art, such as a methylene linker or constrained ethyl linker.

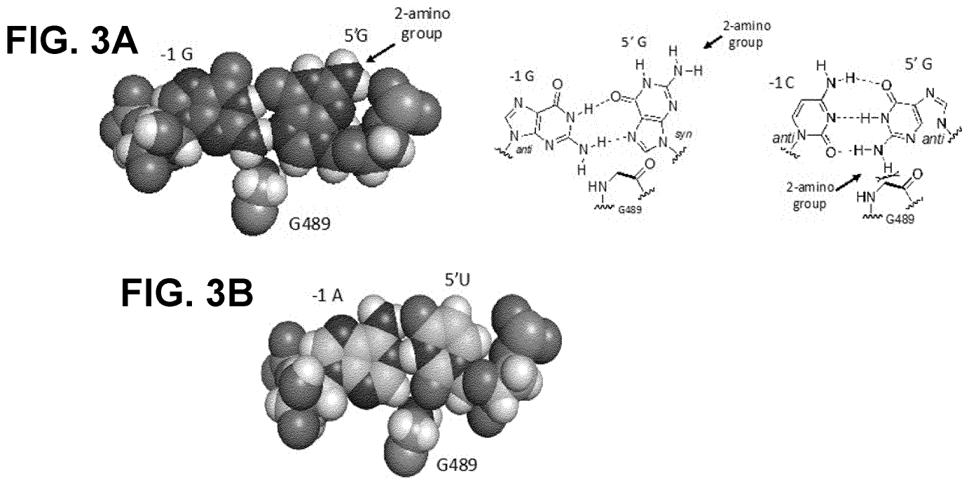

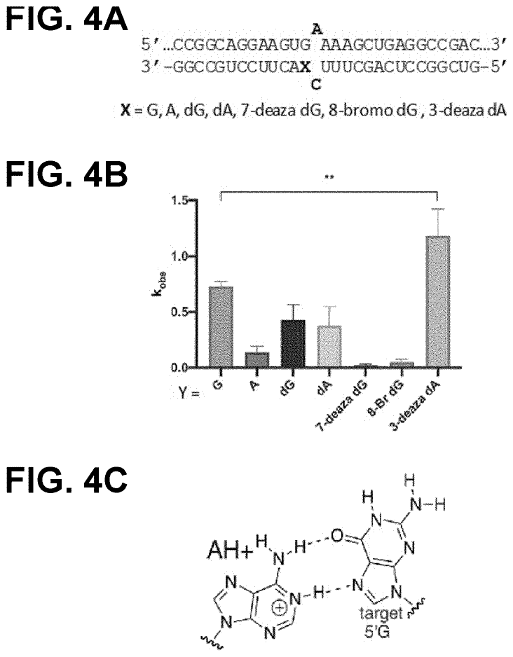

ADARs are multidomain proteins with N-terminal double stranded RNA binding domains (dsRBDs) and C-terminal deaminase domains. Two ADAR genes encode catalytically active ADARs in humans (ADAR encoding ADAR1 proteins and ADARB1 encoding the ADAR2 protein). ADAR1 is expressed in two protein isoforms (p110 and p150) that differ in their N-terminal structures. Since the substrate for ADARs is an RNA duplex, the enzymes access the reactive adenosine using a base flipping mechanism (Stephens O. M. et al. Biochemistry. 2000. 39(40): 12243-12251). Also, because ADARs require duplex RNA for activity, their reaction can be directed to specific adenosines in different transcripts using complementary guide strands for duplex formation at the target sites. This approach is currently being pursued to develop therapeutic guide strands that recruit ADARs to correct disease-causing mutations in RNA (Qu L. et al. Nat. Biotechnol. 2019. 37(9):1059-1069; Merkle T. Et al. Nat. Biotechnol. 2019. 37(2):133-138; Katrekar D. Et al. Nat. Methods 2019. 16(3):239-242; Monian P. Et al. Nat. Biotechnol. 2022: p. Doi: 10.1038/s41587-022-01225-1). While this approach is promising, ADARs have sequence preferences that make certain adenosines disfavored for reaction, limiting the current scope of this approach. For instance, the nearest neighbor nucleotide preferences for ADARs show a strong bias against reaction at adenosines in 5′-GA sites (Eggington J. M. et al. Nat. Commun. 2011. 2(319):DOI:10.1038/ncomms1324). This preference is explained by structural studies of ADAR2 bound to transition state analog-containing RNA that suggest a clash between the 2-amino group of the 5′-G and G489 of the ADAR2 loop involved in stabilizing the flipped-out conformation required for the adenosine deamination reaction (Matthews et al. 2016). Earlierwork with fusion proteins bearing ADAR deaminase domains indicated that editing efficiency at 5′-GA sites could be improved with a G-A or G-G pair at the 5′ nearest neighbor (Schneider M. F. et al. Nucleic Acids Res. 2014. 42(10):p.e87). However, the basis for this effect has not been reported nor has this effect been established for full length ADARs bearing native dsRBD RNA binding domains. Here, it is shown that G-A and G-G pairs on the 5′ side of an editing site improve editing efficiency compared to a 5′ G-C pair for full length ADAR2 and ADAR1 p110. Using X-ray crystallography, the structure of an active fragment of human ADAR2 bound to duplex RNA bearing a G:G pair adjacent to an editing site was determined. Here, the inventors show that the two guanosines can form a hydrogen bonded G_{syn}:Gₐₙₜᵢ pair (instead of, or next to, the occurrence of Gₐₙₜᵢ:Gₐₙₜᵢ pairing) and the beneficial effect of this pairing is rationalized by comparison to similar structures with U-A and C-G pairs adjacent to ADAR editing sites (see FIGS. 3A-3B). In addition, the inventors report the effect on the ADAR deamination rate of several purine analogs paired with a 5′-G in a target site from the MECP2 transcript where the R255X mutation causes Rett Syndrome (see FIGS. 4A-4C). It is at this stage unknown whether G:G pairs only result in G_{syn}:Gₐₙₜᵢ pairing, or whether both types of pairing can occur when a guanosine is opposite a guanosine. In any case, it was determined that the ADAR enzyme prefers the G_{syn}:Gₐₙₜᵢ pairing since the 2-amino group of the 5′-G in the syn conformation does not clash with the minor groove of the enzyme. Together these results show the use of nucleosides capable of stable pairing with the 5′-G in the syn conformation enables more efficient editing within 5′-GA target sites, which now provides a solution to the problem in deaminating these unfavored editing sites. This provides the possibility of (therapeutic) editing of adenosine targets in a 5′-GA environment, which was an unfavored setting before the present invention.

In certain embodiments, the AON is a (single-stranded) AON that targets a pre-mRNA or an mRNA, wherein the target nucleotide in the target (pre-)mRNA molecule is an adenosine, wherein the AON when hybridized to the target RNA molecule can (as a double stranded complex) recruit an adenosine deaminating enzyme, and wherein then the adenosine is deaminated to an inosine, which is being read as a guanosine by the translation machinery.

In an embodiment, the target adenosine is in a UGA (stop) codon, which is then edited to a UGI codon, which is read as UGG (Trp) by the translation machinery, allowing readthrough towards a full-length protein, or allowing readthrough beyond the wild type stop codon represented by the original UGA codon.

In an embodiment, the target adenosine is in a GAU (Asp) codon, which is then edited to a GIU codon, which is then read as GGU (Gly).

In an embodiment, the target adenosine is in a GAC (Asp) codon, which is then edited to a GIC codon, which is then read as GGC (Gly).

In an embodiment, the target adenosine is the first adenosine in a GAA (Glu) codon, which is then edited to a GIA codon, which is then read as GGA (Gly).

In an embodiment, the target adenosine is in a GAG (Glu) codon, which is then edited to a GIG codon, which is then read as GGG (Gly).

Editing the adenosine in a CGA codon to CGI, which is then read as CGG is of no particular use in respect of the resulting protein function, because wild type and edited codon both code for arginine. The same holds true for the second adenosine in an AGA codon, because AGA and AGG both code for arginine. Similarly, editing the adenosine in a GGA codon would produce a GGG, which is likewise of no particular use, because both these codons code for glycine.

Notably, when the first adenosine in a codon is preceded by a guanosine, which is then the third nucleotide in the preceding codon, the editing of that first adenosine may be of interest.

In an embodiment, the target adenosine is the first nucleotide of a codon and preceded by a guanosine in the codon 5′ of the codon in which the adenosine is located, wherein the target adenosine is in a AUU (lie) codon, which is then edited to a IUU codon, which is then read as GUU (Val).

In an embodiment, the target adenosine is the first nucleotide of a codon and preceded by a guanosine in the codon 5′ of the codon in which the adenosine is located, wherein the target adenosine is in an AUC (lie) codon, which is then edited to a IUC codon, which is then read as GUC (Val).

In an embodiment, the target adenosine is the first nucleotide of a codon and preceded by a guanosine in the codon 5′ of the codon in which the adenosine is located, wherein the target adenosine is the first adenosine in a AUA (lie) codon, which is then edited to a IUA codon, which is then read as GUA (Val).

In an embodiment, the target adenosine is the first nucleotide of a codon and preceded by a guanosine in the codon 5′ of the codon in which the adenosine is located, wherein the target adenosine is in an AUG (Met) codon, which is then edited to a IUG codon, which is then read as GUG (Val).

In an embodiment, the target adenosine is the first nucleotide of a codon and preceded by a guanosine in the codon 5′ of the codon in which the adenosine is located, wherein the target adenosine is in an ACU (Thr) codon, which is then edited to an ICU codon, which is then read as GCU (Ala).

In an embodiment, the target adenosine is the first nucleotide of a codon and preceded by a guanosine in the codon 5′ of the codon in which the adenosine is located, wherein the target adenosine is in an ACC (Thr) codon, which is then edited to an ICC codon, which is then read as GCC (Ala).

In an embodiment, the target adenosine is the first nucleotide of a codon and preceded by a guanosine in the codon 5′ of the codon in which the adenosine is located, wherein the target adenosine is the first adenosine in an ACA (Thr) codon, which is then edited to an ICA codon, which is then read as GCA (Ala).

In an embodiment, the target adenosine is the first nucleotide of a codon and preceded by a guanosine in the codon 5′ of the codon in which the adenosine is located, wherein the target adenosine is in an ACG (Thr) codon, which is then edited to an ICG codon, which is then read as GCG (Ala).

In an embodiment, the target adenosine is the first nucleotide of a codon and preceded by a guanosine in the codon 5′ of the codon in which the adenosine is located, wherein the target adenosine is the first adenosine in an AAU (Asn) codon, which is then edited to an IAU codon, which is then read as GAU (Asp).

In an embodiment, the target adenosine is the first nucleotide of a codon and preceded by a guanosine in the codon 5′ of the codon in which the adenosine is located, wherein the target adenosine is the first adenosine in an AAC (Asn) codon, which is then edited to an IAC codon, which is then read as GAC (Asp).

In an embodiment, the target adenosine is the first nucleotide of a codon and preceded by a guanosine in the codon 5′ of the codon in which the adenosine is located, wherein the target adenosine is the first adenosine in an AAA (Lys) codon, which is then edited to an IAA codon, which is then read as GAA (Glu).

In an embodiment, the target adenosine is the first nucleotide of a codon and preceded by a guanosine in the codon 5′ of the codon in which the adenosine is located, wherein the target adenosine is the first adenosine in an AAG (Lys) codon, which is then edited to an IAU codon, which is then read as GAG (Glu).

In an embodiment, the target adenosine is the first nucleotide of a codon and preceded by a guanosine in the codon 5′ of the codon in which the adenosine is located, wherein the target adenosine in an AGU (Ser) codon, which is then edited to an IGU codon, which is then read as GGU (Gly).

In an embodiment, the target adenosine is the first nucleotide of a codon and preceded by a guanosine in the codon 5′ of the codon in which the adenosine is located, wherein the target adenosine in an AGC (Ser) codon, which is then edited to an IGC codon, which is then read as GGC (Gly).

In an embodiment, the target adenosine is the first nucleotide of a codon and preceded by a guanosine in the codon 5′ of the codon in which the adenosine is located, wherein the target adenosine is the first adenosine in an AGA (Arg) codon, which is then edited to an IGA codon, which is then read as GGA (Gly).

In an embodiment, the target adenosine is the first nucleotide of a codon and preceded by a guanosine in the codon 5′ of the codon in which the adenosine is located, wherein the target adenosine in an AGG (Arg) codon, which is then edited to an IGG codon, which is then read as GGG (Gly).

In summary, all the following editing events, wherein X=A/G/C/U, are embodied by the present invention:

5′-...X-UGA-...- 3′ (stop) to 5′-...X-UGG-...- 3′ (Trp)
5′-...X-GAU-...- 3′ (Asp) to 5′-...X-GGU-...- 3′ (Gly)
5′-...X-GAC-...- 3′ (Asp) to 5′-...X-GGC-...- 3′ (Gly)
5′-...X-GAA-...- 3′ (Glu) to 5′-...X-GGA-...- 3′ (Gly)
5′-...X-GAG-...- 3′ (Glu) to 5′-...X-GGG-...- 3′ (Gly)
5′-...G-AUU-...- 3′ (Ile) to 5′-...G-GUU-...- 3′ (Val)
5′-...G-AUC-...- 3′ (Ile) to 5′-...G-GUC-...- 3′ (Val)
5′-...G-AUA-...- 3′ (Ile) to 5′-...G-GUA-...- 3′ (Val)
5′-...G-AUG-...- 3′ (Met) to 5′-...G-GUG-...- 3′ (Val)
5′-...G-ACU-...- 3′ (Thr) to 5′-...G-GCU-...- 3′ (Ala)
5′-...G-ACC-...- 3′ (Thr) to 5′-...G-GCC-...- 3′ (Ala)
5′-...G-ACA-...- 3′ (Thr) to 5′-...G-GCA-...- 3′ (Ala)
5′-...G-ACG-...- 3′ (Thr) to 5′-...G-GCG-...- 3′ (Ala)
5′-...G-AAU-...- 3′ (Asn) to 5′-...G-GAU-...- 3′ (Asp)
5′-...G-AAC-...- 3′ (Asn) to 5′-...G-GAC-...- 3′ (Asp)
5′-...G-AAA-...- 3′ (Lys) to 5′-...G-GAA-...- 3′ (Glu)
5′-...G-AAG-...- 3′ (Lys) to 5′-...G-GAG-...- 3′ (Glu)
5′-...G-AGU-...- 3′ (Ser) to 5′-...G-GGU-...- 3′ (Gly)
5′-...G-AGC-...- 3′ (Ser) to 5′-...G-GGC-...- 3′ (Gly)
5′-...G-AGA-...- 3′ (Arg) to 5′-...G-GGA-...- 3′ (Gly)
5′-...G-AGG-...- 3′ (Arg) to 5′-...G-GGG-...- 3′ (Gly)

The skilled person understands that the sequence in the left column above may be the result of a mutation, which does not necessarily have to be a (point) mutation of the adenosine that is targeted for editing. The codon may be the result of another (point) mutation within the same codon but also an inclusion of nucleotides or even complete codons. Importantly, the editing events as shown above also do not have to be for the purpose of repairing a mutation but may also be applied for gain of function or loss of function purposes. Changing Asp is useful for changing signal sequence sites (to prevent entry into the secretory pathway and/or plasma membrane trafficking and/or secretion) or caspase cleavage sites (to prevent cleavage by caspases) to give an example. Changing Ser and Thr may be useful to prevent phosphorylation of these residues; in some cases, where these residues are so-called master regulators of phosphorylation, this may have major (desirable) effects on protein phosphorylation patterns of the protein in question. Changing a Lys is useful to prevent sumoylation, thereby preventing degradation of a protein.

Making amino acid changes, including the ones described here, may be useful to modulate protein-protein interactions with major impact on protein localization, activation, or function of the protein in question or its interaction partner, potentially affecting entire pathways.

Many of these kinds of changes and their effect on protein interactions or post-translational modifications are known from the literature and specialized databases and the person skilled in the art would readily know how to make these changes through RNA editing. The solutions provided by the present invention related to targeted A-to-I editing where the target adenosine has a 5′-G as neighbor open the possibility to make many more mutation restorations or mutation alleviations, as well as de novo changes with beneficial effects, with efficiencies that are fit for use in a therapeutic setting.

A few, non-limiting examples of mutations that can be targeted by the AONs of the present invention include those observed in:
breast cancer (transcript of BRCA1): Trp>stop (UGG>UGA; rs80356914);
lung cancer (transcript of PPP2R1 B): Gly>Asp (GGC>GAC; rs1805076);
Alpha-1 Antitrypsin deficiency (transcript of SERPINA1): Trp>stop (UGG>UGA; rs1445192595);
Usher syndrome (transcript of USH2A): Trp>stop (UGG>UGA; rs1461319754);
Rett syndrome (transcript of MECP2): R255X (CGA>UGA), R168X (CGA>UGA), and R270X (CGA>UGA);
Haemophilia A (transcript of F8): Gly>Glu (GGA>GAA; rs137852398);
Stargardt disease (transcript of ABCA4): G1961E (GGA>GAA);
Familial Hypercholesteremia Type 1 (transcript of LDLR): Trp>stop (UGG>UGA; rs199570811);
Epileptic Encephalopathy 44 (transcript of UBA5): Ala>Thr (GCA>ACA; rs114925667; target adenosine (underlined) is the first nucleotide in the codon and is preceded by a 5′-G in the preceding codon);
Cystic fibrosis (transcript of CFTR): G551 D (GGU>GAU) and G85E (GGA>GAA);
Marfan Syndrome (transcript of FBN1): Gly>Asp (GGC>GAC; rs7897068);
Gaucher's Disease (transcript of GBA): Gly>Glu (GGG>GAG; rs77829017);
Hereditary Cancer predisposing Syndrome (transcript of MUTYH): Gly>Asp (GGU>GAU; rs36053993) and
Parkinson's disease (transcript of PRKN): G430D (GGC>GAC).

The invention relates to AONs that can bring about efficient deamination, through the adenosine deaminating enzyme, of the target adenosine in the target RNA molecule, wherein the nucleotide that is directly 5′ of the target adenosine is a guanosine (herein often referred to as the 5′-G nucleotide).

The invention relates to an AON that can form a double stranded nucleic acid complex with a target RNA molecule, wherein the double stranded nucleic acid complex can recruit an adenosine deaminating enzyme for deamination of a target adenosine in the target RNA molecule, wherein the nucleotide directly 5′ of the target adenosine in the target RNA molecule is a guanosine, and wherein the nucleotide in the AON that is opposite the guanosine is a nucleotide analog that can induce a syn conformation of the guanosine. In one embodiment, the RNA editing efficiency that is observed with an AON according to the invention, and in which the nucleotide analog opposite the 5′-G has caused the syn conformation of the 5′-G, is higher than an identical AON in which the nucleotide opposite the 5′-G is a guanosine (G) or a deoxyguanosine (dG), see FIG. 4C herein. The skilled person can easily, based on the teaching herein, determine whether a nucleotide analog opposite the 5′-G in a target sequence is suitable for bringing about RNA editing when hybridized to the target sequence, and whether it outperforms an identical AON that carries a guanosine (G) or deoxyguanosine (dG) at that position. As shown herein, the nucleotide analogs as exemplified by 7-deaza dA, 3,7-deaza dA and 3-deaza dA serve that purpose well.

In an embodiment, the nucleotide analog adopts an anti-conformation when the AON is in the double stranded nucleic acid complex with the target RNA molecule. In an embodiment, the nucleotide analog can induce hydrogen bonding to the Hoogsteen face of the 5′-G.

In one embodiment, at least one nucleotide in the AON comprises an ara-ribose. In one embodiment, at least one nucleotide in the AON is a Xeno Nucleic Acid (XNA), such as a bridged nucleic acid (e.g. Locked Nucleic Acid (LNA), constrained ethyl (cEt), and amido-bridged nucleic acid (AmNA)), a tricyclo DNA (tcDNA), an alpha-anomeric bicyclic DNA (abcDNA), a 2′-Fluoro-Arabino Nucleic Acid (FANA), a Glycol Nucleic Acid (GNA), a Threose Nucleic Acid (TNA), a 1,5-anhydrohexitol Nucleic Acid (HNA), a Cyclohexene Nucleic Acid (CeNA), a phosphorodiamidate morpholino (PMO), a L-acyclic threoninol nucleic acid (L-aTNA), a D-acyclic threoninol nucleic acid (D-aTNA), an unlocked nucleic acid (UNA), a serinol nucleic acid (SNA) and a Peptide Nucleic Acid (PNA); or derivatives thereof. In one embodiment, the AON comprises one or more combinations of these XNA's.

The AON according to the invention can comprise internucleoside linkage modifications. In one embodiment one such other internucleoside linkage can be a phosphonoacetate, phosphorothioate (PS), a methylphosphonate (MP), a guanidine phosphoramidate linkage, or a sulfonylphosphoramidate (such as e.g., mesyl phosphoramidate, or 4-acetamidobenzenesulfonyl phosphoramidate). A preferred linkage is a PS linkage. Preferred positions for MP linkages are described in WO2020/201144. In one embodiment, the internucleotide linkage can be a phosphodiester wherein the OH group of the phosphodiester has been replaced by alkyl, alkoxy, aryl, alkylthio, acyl, -NR1R1, alkenyloxy, alkynyloxy, alkenylthio, alkynylthio, —S—Z+, —Se—Z+, or —BH3-Z+, and wherein R1 is independently hydrogen, alkyl, alkenyl, alkynyl, or aryl, and wherein Z+ is ammonium ion, alkylammonium ion, heteroaromatic iminium ion, or heterocyclic iminium ion, any of which is primary, secondary, tertiary or quaternary, or Z is a monovalent metal ion, and is preferably a PS linkage. In one embodiment, the AON comprises stereodefined (also referred to as chirally defined) linkages, including the ones described in WO2011/005761, WO2014/010250, WO2014/012081, WO2015/107425, WO2017/015575, WO2017/062862, WO2017/160741, WO2017/192664, WO2017/192679, WO2017/198775, WO2017/210647, WO2018/067973, WO2018/098264, WO2018/223056, WO2018/223073, WO2018/223081, WO2018/237194, WO2019/032607, WO2019/055951, WO2019/075357, WO2019/200185, WO2019/217784, WO2019/219581, WO2020/118246, WO2020/160336, WO2020/191252, WO2020/196662, WO2020/219981, WO2020/219983, WO2020/227691, WO2021/071788, WO2021/071858, WO2021/178237, WO2021/234459, WO2021/237223, and WO2022/099159.

In the AON of the present invention, the orphan nucleotide (the nucleotide directly opposite the target adenosine) generally comprises a ribose with a 2′—OH group, a deoxyribose with a 2′-H group, a deoxyribose with a 2′—F group (2′-F), an arabinose, a 2′-deoxy-2′-fluoroarabinose (FANA), or a deoxyribose with a 2′,2′-difluoro group, and preferably does not comprise a ribose carrying a 2′-O-Me modification or a 2′-MOE modification. Further, the AON of the present invention generally does not comprise 2′-MOE modifications at certain positions relative to the orphan nucleotide, and further may comprise 2′-MOE and/or 2′-F modifications at other positions within the AON.

In one embodiment, the invention relates to a method for the deamination of at least one target adenosine present in a target RNA molecule in a cell, the method comprising the steps of providing the cell with an AON according to a first aspect of the invention, or a composition according to a second aspect of the invention, allowing uptake by the cell of the AON, allowing annealing of the AON to the target RNA molecule, allowing a mammalian enzyme with nucleotide deaminase activity to deaminate the target nucleotide in the target RNA molecule, and optionally identifying the presence of the deaminated nucleotide in the target RNA molecule. Preferably, the presence of the target RNA molecule is detected by either (i) sequencing the target sequence, (ii) assessing the presence of a functional, elongated, full length and/or wild type protein when the target adenosine is located in a UGA stop codon, which is edited to a UGG codon through the deamination, or (iii) using a functional read-out, wherein the target RNA after the deamination encodes a functional, full length, elongated and/or wild type protein. The present invention therefore also relates to AONs that target premature termination stop codons (PTCs) present in the (pre-)mRNA to alter the adenosine present in the stop codon to an inosine (read as a G), which in turn then results in read-through during translation and a full-length functional protein.

The teaching of the present invention, as outlined herein, is applicable for all genetic diseases that may be targeted with AONs and may be treated through RNA editing. However, it is also applicable to ‘loss of function’ editing, or in another embodiment a ‘gain-of-function’ editing in which the deamination of the adenosine causes a gain of function, for instance the introduction of another secondary protein structure or the introduction of a functional site, for instance a phosphorylation site. In any case, the invention relates to AONs that can be used to edit adenosines that have a 5′ neighbouring guanosine.

In one embodiment, the AON according to the invention comprises 2, 3, 4, 5, 6, 7, 8, 9 or 10 mismatches, wobbles and/or bulges with the complementary target RNA region. When the nucleotide opposite the target adenosine is a cytidine (generally deoxycytidine (dC), or a 2′-deoxycytidine carrying another substitute than 2′-O-Me or 2′-MOE, as further outlined herein), or a cytidine analog as for example disclosed in WO2020/252376, the AON mismatches at least once with the target RNA molecule. However, the AON according to the invention and as outlined herein in detail, the 5′-G in the target sequence is opposite a nucleotide analog which does not have a Watson/Crick base pairing with that 5′-G either. However, in certain embodiments it is preferred to position a U opposite the target adenosine, which is in principle not a mismatch. In a preferred aspect one or more additional mismatching nucleotides, wobbles and/or bulges are present between AON and target RNA. These should add to the RNA editing efficiency by the ADAR present in the cell, at the target adenosine position. The person skilled in the art can determine whether hybridization under physiological conditions still does take place. The AON of the present invention can recruit (engage) a mammalian adenosine deaminating enzyme present in the cell. This is preferably an ADAR enzyme, more preferably ADAR1 or ADAR2. However, when the target RNA molecule is a tRNA, the adenosine deaminating enzyme may also be an Adenosine Deaminase acting on tRNA (ADAT). The AONs according to the present invention can utilise endogenous cellular pathways and naturally available ADAR enzymes, or enzymes with ADAR activity (which may be yet unidentified ADAR-like enzymes) to specifically edit a target adenosine in a target RNA sequence. As disclosed herein, the single-stranded AONs of the invention can bring about deamination of a specific target, such as adenosine, in a target RNA molecule, wherein the 5′ neighbouring nucleotide from the adenosine is a guanosine. Ideally, at least one target nucleotide is deaminated. Alternatively, 1, 2, or 3 further nucleotides are deaminated, which do not necessarily have a 5′-GA configuration. This means that a single AON according to the invention may be applied for deamination of a target adenosine that has a 5′-G, but also for deamination further up- or downstream of the target adenosine of another adenosine that may have a 5′-U, a 5′-C, or a 5′-A neighbouring nucleotide. The nucleoside analog according to the present invention, which is opposite the 5′-G in the target RNA molecule, may be within an AON that brings about RNA editing, wherein the AON comprises a stem-loop structure (a self-looping hairpin structure) as disclosed for instance in WO2016/097212, WO2017/010556, WO2017/050306, WO2019/111957, WO2020/001793, WO2021/113270, WO2021/243023, WO2022/078995, or wherein the AON is fully or almost fully complementary to the target RNA molecule's sequence, and does not necessarily comprise a hairpin structure, such as for instance disclosed in WO2017/220751 and WO2018/041973. Notably, the AON of the present invention may also be linked to recombinant deaminase domains, as was shown by Montiel-Gonzalez et al. (2013) and Vogel et al. (2014), because such ‘early’ technologies in which oligonucleotide sequences were used to bring about RNA editing may also benefit from the introduction of a nucleoside analog opposite the 5′-G, as further outlined in the present invention. Hence, the AONs of the present invention are not limited in the sense that the AON does not have a stem-loop structure, or that the AON is not linked to a deaminase or deaminase domain. Nevertheless, it is preferred that the AON is relatively short such that cell entry is feasible and the AON is therapeutically relevant when administered in a ‘naked’ form, and therefore preferably does not have a stem-loop structure and preferably is not linked to deaminase moieties. However, the AON of the present invention may be linked to other moieties that may enhance cellular uptake or cellular trafficking towards the site of action. Examples are oligonucleotides that are conjugated to GaINAc ligands, such as those described in WO2014/179620 and WO2017/079745, which improve the delivery of oligonucleotides in vivo, especially to liver cells. Another example is formed by the group of saponins that can be conjugated with the oligonucleotide, and which improve cellular entry and trafficking (WO2020/126626; WO2021/122998; Wang M. et al. Drug Design, Development and Therapy. 2018. 12:3705-3715). In one embodiment, the AON of the present invention is conjugated to one or more GaINAc ligands and/or to a saponin. The skilled person is aware of what ligands and what saponins may be best used for what therapeutic use.

In one embodiment, the AON capable of forming a double stranded nucleic acid complex with a target RNA molecule, wherein the double stranded nucleic acid complex can recruit an adenosine deaminating enzyme for deamination of at least one target adenosine in the target RNA molecule comprises a cytidine analog that is directly opposite the at least one target adenosine, in which the cytidine analog serves as an H-bond donor at the N3 site. Preferably, the cytidine analog is pseudoisocytidine (piC) or Benner's base Z. These cytidine analog nucleotides can come in an RNA or DNA format, or potentially modified at the 2′ position. Other cytidine analogs that can also be used in oligonucleotides according to the invention are derivatives of pseudoisocytidine (piC), Benner's base Z, 5-hydroxyC-H+, 5-aminoC-H+ and 8-oxoA (syn), such as cytidine C5 methyl, ethyl, propyl, etc., variants of the Benner's base Z that have different substituents than nitro (e.g. alkyl, F, Cl, Br, CN, etc.) and variants of 8-oxoA that are substituted at C2 (methyl, ethyl, propyl, halogens, etc). In one embodiment, the cytidine analog does not carry a 2′-O-Me or 2′-MOE ribose modification.

In one embodiment, the AON that is capable of forming a double stranded nucleic acid complex with a target RNA molecule, wherein the double stranded nucleic acid complex can recruit an adenosine deaminating enzyme for deamination of at least one target adenosine in the target RNA molecule, comprises a uridine analog or uridine derivative that is directly opposite the target adenosine, wherein the uridine analog or uridine derivative serves as an H-bond donor at the N3 site. Examples of preferred uridine analogs and uridine derivatives are iso-uridine, pseudouridine, 4-thiouridine, thienouridine, 5-methoxyuridine, dihydrouridine, 5-methyluridine N3-glycosylated uridine, and dihydro-iso-uridine. These uridine analogs/derivatives can come in an RNA or DNA format or can potentially be modified at the 2′ position. Other uridine analogs that can also be used in oligonucleotides according to the invention are derivatives of iso-uridine, such as substituted iso-uridine variants (with e.g., nitro, alkyl, F, Cl, Br, CN, etc.).

In one embodiment, the AON according to the present invention comprises at least one phosphonoacetate, phosphorothioate (PS), a methylphosphonate (MP), a guanidine phosphoramidate linkage, a phosphoryl guanidine linkage, a phosphoramidate linkage, or a sulfonylphosphoramidate (such as e.g., mesyl phosphoramidate, or 4-acetamidobenzenesulfonyl phosphoramidate). In a preferred embodiment, the double stranded nucleic acid complex can recruit an endogenous ADAR enzyme, preferably wherein the ADAR enzyme is an endogenous ADAR2 enzyme. The double stranded AON/target RNA molecule complex interacts through Watson-Crick base-pairing, except at the position of the 5′-G and generally also not at the position of the target adenosine which is opposite the orphan nucleotide (which may be a nucleotide carrying a Benner's base, a cytidine, a uridine (=not a mismatch), or a uridine analog/derivative as outlined above). The skilled person is able, based on the teaching available in the art to determine the level of capability to achieve RNA editing and compare the capabilities of the chemically modified positions or the full AON to an AON lacking specific sugar- and/or linkage modifications at specified positions. The length of the AON may vary depending on the structures that are present (hairpin structured AONs are generally longer, but when no hairpin structure is present, the AON may be relatively ‘short’, preferably comprising approximately 15 to 30 nucleotides). The AON of the present invention does not necessarily carry a recruiting portion (such as a hairpin or a stem-loop structure) to attract ADAR, but it is not excluded.

The invention relates to an antisense oligonucleotide (AON) capable of forming a double stranded nucleic acid complex with a target RNA molecule, wherein the double stranded nucleic acid complex is capable of recruiting an adenosine deaminating enzyme for deamination of a target adenosine in the target RNA molecule, wherein the nucleotide directly 5′ of the target adenosine in the target RNA molecule is a guanosine, and wherein the nucleotide in the AON that is opposite the guanosine is a nucleotide analog that can induce a syn conformation of the guanosine. Determining whether a 5′-G in a certain target RNA molecule is feasible based on the teaching as outlined herein. Any AON that comprises a nucleotide analog that can induce a syn confirmation of the 5′-G and provides a higher rate of RNA editing in vitro and/or in vivo than an identical AON that comprises an adenosine or guanosine at the position of the nucleotide analog (opposite the 5′-G) is an embodiment of the present invention. The skilled person can determine whether an AON comprising a nucleotide analog, preferably one that comprises a modified purine nucleobase at the position opposite the 5′-G outperforms (works better, more efficient, and/or quicker in respect of bringing about RNA editing) than an AON comprising an adenosine, deoxyadenosine, guanosine, or deoxyguanosine at that same position opposite the 5′-G, based on the teaching herein and based on the common general knowledge within the field of RNA editing. The skilled person can also determine, based on the teaching herein and the general common knowledge in the field of crystallography, and other technologies, whether the 5′-G is brought into a syn conformation when hybridized to the guide AON and in complex with the deaminase domain of the adenosine deaminating enzyme.

In an embodiment, the invention relates to an AON according to the invention, wherein at least one nucleotide or nucleotide analog in the AON comprises a substitution at the 2′ position of the ribose, wherein the substitution is selected from the group consisting of: H (DNA); OH (RNA); F; ara-F; diF; 2‘-C-methyl’-2′-F; substituted or unsubstituted, linear or branched lower (Cl-Cao) alkyl, alkenyl, alkynyl, alkaryl, allyl, or aralkyl, that may be interrupted by one or more heteroatoms; O-, S-, or N-alkyl; O-, S-, or N-alkenyl; O-, S-, or N-alkynyl; O-, S-, or N-allyl; O-alkyl-O-alkyl; methoxy; aminopropoxy; methoxyethoxy; dimethylamino oxyethoxy; and dimethylaminoethoxyethoxy; with the proviso that the nucleotide that is opposite the target adenosine does not comprise a 2′-O-methyl or a 2′-(2-methoxy)ethyl ribose modification. In an embodiment, the invention relates to an AON according to the invention, wherein at least one nucleotide or nucleotide analog in the AON is an arabinonucleic acid. In an embodiment, the invention relates to an AON according to the invention, wherein the nucleotide analog comprises a modified nucleobase. In an embodiment, the invention relates to an AON according to the invention, wherein the nucleotide analog comprises a modified purine nucleobase. In an embodiment, the invention relates to an AON according to the invention, wherein the modified purine nucleobase comprises a 7-deaza-purine modification, preferably a 7-deaza-adenine modification, or a 3-deaza-purine modification, preferably a 3-deaza-adenine modification, or a 3,7-dideaza modification, preferably a 3,7-dideaza-adenine modification. In an embodiment, the invention relates to an AON according to the invention, wherein the modified purine nucleobase comprises a hydrogen bond donor at N1 that has a pKa that is higher than 3.7 and lower than 9.5. In an embodiment, the invention relates to an AON according to the invention, wherein the nucleotide analog is selected from the group consisting of:
7-deaza-2′-deoxyadenosine (7-deaza dA);
7-deaza-2′-adenosine (7-deaza A);
7-deaza-2′-deoxy-2′-fluoroadenosine (7-deaza fA);
7-deaza-2′-deoxy-2′-ara-fluoroadenosine;
7-deaza-2′-deoxy-2′,2′-difluoroadenosine;
3-deaza-2′-deoxyadenosine (3-deaza dA);
3-deaza-2′-adenosine (3-deaza A);
3-deaza-2′-deoxy-2′-fluoroadenosine (3-deaza fA);
3-deaza-2′-deoxy-2′-ara-fluoroadenosine;
3-deaza-2′-deoxy-2′,2′-difluoroadenosine;
3,7-dideaza-2′-deoxyadenosine (3,7-dideaza dA);
3,7-dideaza-2′-adenosine (3,7-dideaza A);
3,7-dideaza-2′-deoxy-2′-fluoroadenosine (3,7-dideaza fA);
3,7-dideaza-2′-deoxy-2′-ara-fluoroadenosine;
3,7-dideaza-2′-deoxy-2′,2′-difluoroadenosine;
3-deaza-2′-[2-(methoxy)ethyl]adenosine;
3-deaza-2′-O-[2-methylamino-2-oxoethyl]adenosine;
2′-deoxy-2′-fluoroguanosine;
2′-ara-fluoro guanosine (FANA G);
2′,2′-difluoro guanosine;
2′-deoxyinosine (dl);
2′-OH-inosine (rl);
2′-fluoroinosine (2′-F-l);
2′-ara-fluoro inosine (FANA I);
2′,2′-difluoro inosine;
5-formylindole-2′-deoxyriboside;
5-formyl-2′-fluoro-2′-deoxyriboside
5-formylindole-2′-ara-fluoro-2′-deoxyriboside;
5-formylindole-2′,2′-difluoro-2′-deoxyriboside;
5-formylindole-2′-O-methylriboside;
5-formylindole-2′-O-[2-(methoxy)ethyl]riboside;
5-formylindole-2′-O-[2-methylamino-2-oxoethyl]riboside
beta-(4-amidino-1H-imidazol-1-yl) riboside;
beta-(4-amidino-1H-imidazol-1-yl) 2′-deoxyriboside;
beta-(4-amidino-1H-imidazol-1-yl) 2′-ara-fluoro-2′-deoxyriboside; and
beta-(4-amidino-1H-imidazol-1-yl) 2′,2′-difluoro-2′-deoxyriboside.

In an embodiment, the nucleotide opposite the 5′-G in the target sequence comprises a base moiety with the structure of formula (II), (III), (IV), (V), (VI), or (VII):

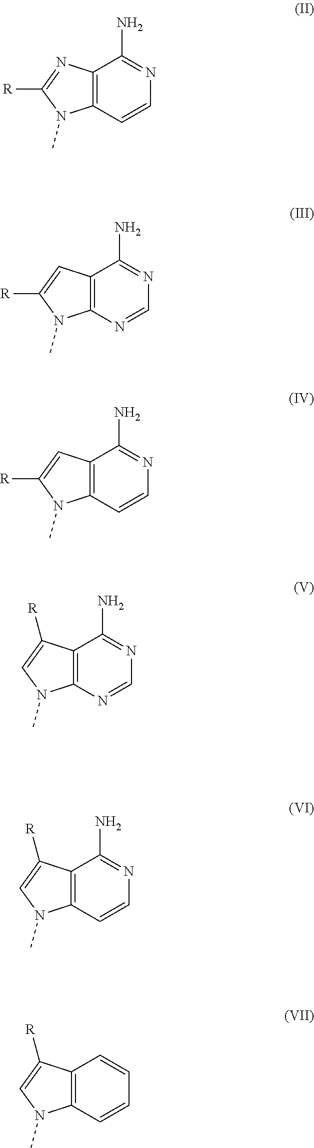

wherein R is:
H;
an electron-donating moiety;
—C(═O)H
—OR₁;
—SR₁; or
—NR₁R₂ wherein R₁ is (C₁-C₆)-alkoxy, methoxy, ethoxy, isopropoxy, cyclopropoxy, (C₁-C₆)-alkyl, methyl, ethyl, isopropyl, or cyclopropyl, and R₂ is H, (C₁-C₆)-alkoxy, methoxy, ethoxy, isopropoxy, cyclopropoxy, (C₁-C₆)-alkyl, methyl, ethyl, isopropyl, or cyclopropyl.

In an embodiment, the nucleotide opposite the 5′-G in the target sequence comprises a base structure according to formula (VIII) or (IX):

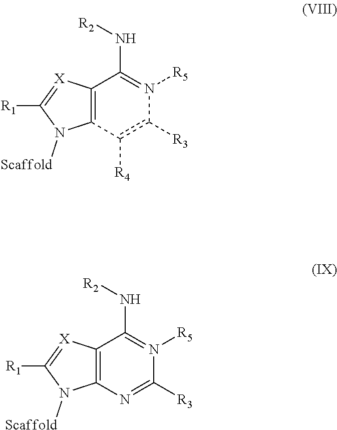

wherein:
X═CH, CR₆ or N;
R₁=H, OH, halogen, SH, (C₁-C₃)-alkoxy, or NH₂;
R₂═H, OH, NH₂, methyl, ethyl, or cyclopropyl;
R₃═H, OH, NH₂, methyl, ethyl, cyclopropyl, or (C₁-C₃)-alkoxy;
R₄═H, OH, NH₂, methyl, ethyl, cyclopropyl, or (C₁-C₃)-alkoxy;
R₅=H, OH, NH₂, methyl, ethyl, cyclopropyl, or (C₁-C₃)-alkoxy; and
R₆=an electron-donating moiety, methyl, ethyl cyclopropyl, or (C₁-C₃)-alkoxy.

In an embodiment, the invention relates to an AON according to the invention, wherein the adenosine deaminating enzyme is an endogenous ADAR enzyme, preferably ADAR2. In an embodiment, the invention relates to an AON according to the invention, wherein the AON comprises at least one phosphorothioate (PS), phosphonoacetate, or a methylphosphonate (MP) internucleotide linkage. In an embodiment, the invention relates to an AON according to the invention, wherein the AON comprises at least 15, 16, 17, 18, 19, 20, 21, 22, 23, 24, 25, 26, 27, 28, 29, 30, 31, 32, 33, 34, 35, or 36 nucleotides, and is at most 100 nucleotides long. A preferred length of the AON according to the invention is approximately 15 to approximately 25 nucleotides.

In another embodiment, the AON according to the invention does not comprise a sequence that allows the formation of an internal stem-loop structure (or hairpin structure). In another preferred embodiment, the AON according to the invention is not linked, covalently, or non-covalently to a (recombinant) deaminase enzyme or a (recombinant) deaminase domain thereof.

In an embodiment, the invention relates to a pharmaceutical composition comprising an AON according to the invention, and a pharmaceutically acceptable carrier or diluent. Pharmaceutically acceptable carriers or diluent are well known in the art. In an embodiment, the invention relates to an AON according to the invention, or a pharmaceutical composition according to the invention, for use in the treatment, amelioration, or slowing down progression of a genetic disease caused by a premature UGA termination codon.

In an embodiment, the invention relates to a method for the deamination of at least one target adenosine present in a target RNA molecule in a cell, wherein the nucleotide 5′ of the target adenosine in the RNA molecule is a guanosine, the method comprising the steps of: (i) providing the cell with an AON according to the invention, or a pharmaceutical composition according to the invention; (ii) allowing annealing of the AON to the target RNA molecule to form a double stranded nucleic acid complex capable of recruiting an adenosine deaminating enzyme in the cell, preferably an endogenous adenosine deaminating enzyme; (iii) allowing the adenosine deaminating enzyme to deaminate the target adenosine in the target RNA molecule; and (iv) optionally identifying the presence of the deaminated adenosine in the target RNA molecule. In an embodiment, step (iv) comprises: (a) sequencing a region of the target RNA molecule, wherein the region comprises the deaminated target adenosine; (b) assessing the presence of a functional, elongated, full length and/or wild type protein when the target adenosine is in a UGA stop codon; or (c) using a functional read-out, wherein the target RNA molecule after the deamination encodes a functional, full length, elongated and/or wild type protein.

In an embodiment, the invention relates to a method for the deamination of at least one target adenosine present in a target RNA molecule, the method comprising the steps of: (i) providing an AON according to the invention, or a pharmaceutical composition according to the invention; (ii) allowing annealing of the AON to the target RNA molecule to form a double stranded nucleic acid complex with the target RNA molecule; (iii) allowing a mammalian adenosine deaminating enzyme to deaminate the target adenosine in the target RNA molecule; and (iv) optionally identifying the presence of the deaminated adenosine in the target RNA molecule. Preferably, the adenosine deaminating enzyme is an endogenous ADAR enzyme, preferably ADAR2.

In an embodiment, the invention relates to an AON according to the invention, or a pharmaceutical composition according to the invention, for use in the treatment of a genetic disorder, preferably selected from the group consisting of: Hurler Syndrome, alpha-1-antitrypsin (A1AT) deficiency, (familial) hypercholesterolemia, Parkinson's disease, Rett syndrome, Stargardt Disease, Citrullinemia Type 1, autosomal recessive non-syndromic hearing loss, X-linked retinoschisis, argininosuccinate lyase deficiency, Duchenne/Becker muscular dystrophy, Non-Alcoholic Steatohepatitis (NASH), Myotonic dystrophy type I, Myotonic dystrophy type II, Huntington's disease, Usher syndrome (such as Usher syndrome type I, II, and Ill), Charcot-Marie-Tooth disease, Cystic fibrosis, Alzheimer's disease, albinism, Amyotrophic lateral sclerosis, Asthma, B-thalassemia, Epileptic Encephalopathy, CADASIL syndrome, Chronic Obstructive Pulmonary Disease (COPD), Distal Spinal Muscular Atrophy (DSMA), Dystrophic Epidermolysis bullosa, Epidermolysis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hereditary Cancer predisposing Syndrome, Hunter Syndrome, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, neurofibromatosis, Niemann-Pick disease type A, B and C, NY-eso1 related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, (autosomal dominant) Retinitis Pigmentosa, Sandhoff Disease, Severe Combined Immune Deficiency Syndrome (SCID), Sickle Cell Anaemia, Spinal Muscular Atrophy, Tay-Sachs Disease, X-linked immunodeficiency, Sturge-Weber Syndrome, and cancer, such as breast and lung cancer.

In an embodiment, the invention relates to a method of treating, preventing, or ameliorating a genetic disorder, comprising the step of administering an AON according to the invention to a subject suffering from said genetic disorder. In an embodiment, the genetic disorder is caused by a mutation resulting in a premature termination codon, wherein said premature termination codon is UGA. In another embodiment, the genetic disorder is not the result of a premature termination stop codon, but may be the result of a mutation resulting in an unwanted and disease-causing codon, wherein the codon comprises a target adenosine, which has a preceding 5′-G, either in the codon of the target adenosine itself or wherein the 5′-G is the third nucleotide of the codon that is 5′ of the codon in which the target adenosine is the first nucleotide. In an embodiment the genetic disorder is selected from the group consisting of: Hurler Syndrome, alpha-1-antitrypsin (A1AT) deficiency, (familial) hypercholesterolemia, Parkinson's disease, Rett syndrome, Stargardt Disease, Citrullinemia Type 1, autosomal recessive non-syndromic hearing loss, X-linked retinoschisis, argininosuccinate lyase deficiency, Duchenne/Becker muscular dystrophy, Non-Alcoholic Steatohepatitis (NASH), Myotonic dystrophy type I, Myotonic dystrophy type II, Huntington's disease, Usher syndrome (such as Usher syndrome type I, II, and Ill), Charcot-Marie-Tooth disease, Cystic fibrosis, Alzheimer's disease, albinism, Amyotrophic lateral sclerosis, Asthma, B-thalassemia, Epileptic Encephalopathy, CADASIL syndrome, Chronic Obstructive Pulmonary Disease (COPD), Distal Spinal Muscular Atrophy (DSMA), Dystrophic Epidermolysis bullosa, Epidermolysis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hereditary Cancer predisposing Syndrome, Hunter Syndrome, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, neurofibromatosis, Niemann-Pick disease type A, B and C, NY-eso1 related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, (autosomal dominant) Retinitis Pigmentosa, Sandhoff Disease, Severe Combined Immune Deficiency Syndrome (SCID), Sickle Cell Anaemia, Spinal Muscular Atrophy, Tay-Sachs Disease, X-linked immunodeficiency, Sturge-Weber Syndrome, and cancer, such as breast and lung cancer.

In yet another embodiment, the invention relates to an AON capable of forming a double stranded nucleic acid complex with a target RNA molecule, for use in the treatment of a genetic disorder, wherein the double stranded nucleic acid complex is capable of recruiting an adenosine deaminating enzyme for deamination of a target adenosine in the target RNA molecule, wherein the nucleotide directly 5′ of the target adenosine in the target RNA molecule is a guanosine (5′-G), and wherein the nucleotide in the AON that is opposite the guanosine is a natural nucleotide selected from the group consisting of: guanosine, deoxyguanosine, adenosine, deoxyadenosine, and inosine; wherein the natural nucleotide can induce a syn conformation of the 5′-G. In a preferred embodiment of the AON that is for use in the treatment of a genetic disorder according to the invention, the genetic disorder is selected from the group consisting of: Hurler Syndrome, alpha-1-antitrypsin (A1AT) deficiency, (familial) hypercholesterolemia, Parkinson's disease, Rett syndrome, Stargardt Disease, Citrullinemia Type 1, autosomal recessive non-syndromic hearing loss, X-linked retinoschisis, argininosuccinate lyase deficiency, Duchenne/Becker muscular dystrophy, Non-Alcoholic Steatohepatitis (NASH), Myotonic dystrophy type I, Myotonic dystrophy type II, Huntington's disease, Usher syndrome (such as Usher syndrome type I, II, and Ill), Charcot-Marie-Tooth disease, Cystic fibrosis, Alzheimer's disease, albinism, Amyotrophic lateral sclerosis, Asthma, ß-thalassemia, Epileptic Encephalopathy, CADASIL syndrome, Chronic Obstructive Pulmonary Disease (COPD), Distal Spinal Muscular Atrophy (DSMA), Dystrophic Epidermolysis bullosa, Epidermolysis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hereditary Cancer predisposing Syndrome, Hunter Syndrome, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, neurofibromatosis, Niemann-Pick disease type A, B and C, NY-eso1 related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, (autosomal dominant) Retinitis Pigmentosa, Sandhoff Disease, Severe Combined Immune Deficiency Syndrome (SCID), Sickle Cell Anaemia, Spinal Muscular Atrophy, Tay-Sachs Disease, X-linked immunodeficiency, Sturge-Weber Syndrome, and cancer, such as breast and lung cancer.

In yet another embodiment, the invention relates to an AON capable of forming a double stranded nucleic acid complex with a target RNA molecule, for use in the treatment of a genetic disorder, wherein the double stranded nucleic acid complex is capable of recruiting an adenosine deaminating enzyme for deamination of a target adenosine in the target RNA molecule, wherein the nucleotide directly 5′ of the target adenosine in the target RNA molecule is a guanosine (5′-G), and wherein the nucleotide in the AON that is opposite the guanosine is a natural nucleotide or a nucleotide analog as outlined herein, wherein the natural nucleotide or nucleotide analog can induce a syn conformation of the 5′-G, and wherein the genetic disorder is not caused by a mutation, but wherein the use is for a gain-of-function purpose to alleviate, treat, prevent, or ameliorate a disease.

The (mammalian) enzyme with nucleotide deaminase, and preferably adenosine deamination activity that is engaged using the AON according to the invention is preferably an ADAR enzyme, more preferably ADAR1 or ADAR2, even more preferably an endogenous ADAR1 or ADAR2 enzyme present in a cell and can alter the target nucleotide in the target RNA molecule, which target nucleotide is then preferably an adenosine that is deaminated to an inosine. Other adenosine deaminating enzymes that may be recruited by the AON of the present invention is the Adenosine Deaminating enzyme Acting on tRNA (ADAT), which is preferably also an endogenous, and more preferably an endogenous human ADAT.

In another embodiment, the invention relates to a method of treating a subject, preferably a human subject in need thereof, wherein the subject suffers from a genetic disorder caused by a mutation resulting in a premature termination codon such as UGA, which contains the unfavoured 5′-GA sequence as discussed above.

Even though the mutation may not be G>A mutation (from wt UGG; tryptophan) but for instance a C>G (from wt UCA; serine), U>G (from wt UUA; leucine), C>A (from wt UGC; cysteine), or U>A (from wt UGU; cysteine) mutation, the deamination of the adenosine in this codon may still be beneficial for the patient suffering from a disease caused by the occurrence of the UGA premature termination codon. Deamination of the target adenosine to an inosine would alleviate, prevent, or ameliorate the disease, even though the resulting codon is read as UGG which may be different from the wild-type codon (see above, except when the UGA codon is the result of a G>A mutation). The fact that the protein will be produced in its full length is generally believed to be beneficial, which can further be substantiated by experiments known to the person skilled in the art. Clearly, any adenosine that is 3′ from a guanosine in the target sequence may now be deaminated using the teaching of the present invention. The target adenosine may be the first, second, or third nucleotide of a codon, as long as the preceding nucleotide of the adenosine is a guanosine, which may be then the third, first or second nucleotide of a codon, respectively. Any type of surrounding sequence around the 5′-GA configuration is therefore potentially allowed.

In one embodiment, a method comprises the steps of administering to the subject an AON or pharmaceutical composition according to the invention, allowing the formation of a double stranded nucleic acid complex of the AON with its specific complementary target nucleic acid in a cell in the subject; allowing the engagement of an endogenous present adenosine deaminating enzyme, such as ADAR2; and allowing the enzyme to deaminate the target adenosine in the target nucleic target molecule to an inosine, thereby alleviating, preventing or ameliorating the genetic disease. The genetic diseases that may be treated according to this method are preferably, but not limited to the genetic diseases listed herein, and any other disease in which deamination of a specific adenosine would be beneficial for a patient in need thereof.

The skilled person knows that an oligonucleotide, such as an RNA oligonucleotide, generally consists of repeating monomers. Such a monomer is most often a nucleotide or a chemically modified nucleotide. The most common naturally occurring nucleotides in RNA are adenosine monophosphate (A), cytidine monophosphate (C), guanosine monophosphate (G), and uridine monophosphate (U). These consist of a pentose sugar, a ribose, a 5′-linked phosphate group which is linked via a phosphate ester, and a 1′-linked base. The sugar connects the base and the phosphate and is therefore often referred to as the “scaffold” of the nucleotide. A modification in the pentose sugar is therefore often referred to as a “scaffold modification”. The original pentose sugar may be replaced in its entirety by another moiety that similarly connects the base and the phosphate. It is therefore understood that while a pentose sugar is often a scaffold, a scaffold is not necessarily a pentose sugar. Examples of scaffold modifications that may be applied in the monomers of the AONs of the present invention are disclosed in WO2020/154342, WO2020/154343, and WO2020/154344. A base, sometimes called a nucleobase, is generally adenine, cytosine, guanine, thymine or uracil, or a derivative thereof. A base, sometimes called a nucleobase, is defined as a moiety that can bond to another nucleobase through H-bonds, polarized bonds (such as through CF moieties) or aromatic electronic interactions. Cytosine, thymine, and uracil are pyrimidine bases, and are generally linked to the scaffold through their 1-nitrogen. Adenine and guanine are purine bases and are generally linked to the scaffold through their 9-nitrogen.

A nucleotide is generally connected to neighboring nucleotides through condensation of its 5′-phosphate moiety to the 3′-hydroxyl moiety of the neighboring nucleotide monomer. Similarly, its 3′-hydroxyl moiety is generally connected to the 5′-phosphate of a neighboring nucleotide monomer. This forms phosphodiester bonds. The phosphodiesters and the scaffold form an alternating copolymer. The bases are grafted on this copolymer, namely to the scaffold moieties. Because of this characteristic, the alternating copolymer formed by linked scaffolds of an oligonucleotide is often called the “backbone” of the oligonucleotide. Because phosphodiester bonds connect neighboring monomers together, they are often referred to as “backbone linkages”. It is understood that when a phosphate group is modified so that it is instead an analogous moiety such as a phosphorothioate (PS), such a moiety is still referred to as the backbone linkage of the monomer. This is referred to as a “backbone linkage modification”. In general terms, the backbone of an oligonucleotide comprises alternating scaffolds and backbone linkages.

The nucleobases in an AON of the present invention can be adenine, cytosine, guanine, thymine, or uracil or any other moiety able to interact with another nucleobase through H-bonds, polarized bonds (such as CF) or aromatic electronic interactions. Clearly, the nucleobase (in an AON of the present invention) that is opposite the 5′-G in the target sequence is a modified nucleobase, also referred to as a “base analog”. The entire monomer or nucleotide may be referred to as a “nucleotide analog”. Any analog at this position in the AON of the invention is suitable if it can bring the 5′-G in a syn conformation and allows more efficient RNA editing brought about by the AON in complex with its target sequence than is observed when an identical AON is used but wherein there is a‘normal’ guanosine or deoxyguanosine opposite the 5′-G, as explained in the accompanying examples. Hence, any modification of the scaffold or backbone may be introduced within the AON and also at the position of the nucleotide analog opposite the 5′-G as long as the AON does not suffer in RNA editing efficiency. This means that the ribose may be further chemically modified at the 1′, 2′, 3′, 4′, and/or 5′ position(s) and the skilled person can introduce such modifications at will and still be able to monitor the effect of the base analog in its interaction with the 5′-G in the target sequence.

The nucleobases at any position in the AON of the present invention can be a modified form of adenine, cytosine, guanine, or uracil, such as hypoxanthine (the nucleobase in inosine), isouracil, pseudouracil, pseudocytosine, 1-methylpseudouracil, orotic acid, agmatidine, lysidine, 2-thiouracil, 2-thiothymine, 5-halouracil, 5-halomethyluracil, 5-trifluoromethyluracil, 5-propynyluracil, 5-propynylcytosine, 5-aminomethyluracil, 5-hydroxymethyluracil, 5-formyluracil, 5-aminomethylcytosine, 5-formylcytosine, 5-hydroxymethylcytosine, 7-deazaguanine, 7-deazaadenine, 7-deaza-2,6-diaminopurine, 8-aza-7-deazaguanine, 8-aza-7-deazaadenine, 8-aza-7-deaza-2,6-diaminopurine, pseudoisocytosine, N4-ethylcytosine, N2-cyclopentylguanine, N2-cyclopentyl-2-aminopurine, N2-propyl-2-aminopurine, 2,6-diaminopurine, 2-aminopurine, G-clamp, Super A, Super T, Super G, amino-modified nucleobases or derivatives thereof; and degenerate or universal bases, like 2,6-difluorotoluene, or absent like abasic sites (e.g. 1-deoxyribose, 1,2-dideoxyribose, 1-deoxy-2-O-methylribose, azaribose). The terms ‘adenine’, ‘guanine’, ‘cytosine’, ‘thymine’, ‘uracil’ and ‘hypoxanthine’ as used herein refer to the nucleobases as such. The terms ‘adenosine’, ‘guanosine’, ‘cytidine’, ‘thymidine’, ‘uridine’ and ‘inosine’ refer to the nucleobases linked to the (deoxy)ribosyl sugar.

In an embodiment, the nucleotide analog is an analog of a nucleic acid nucleotide. In an embodiment, the nucleotide analog is an analog of adenosine, guanosine, cytidine, thymidine, uridine, deoxyadenosine, deoxyguanosine, deoxycytidine, deoxythymidine or deoxyuridine. In an embodiment, the nucleotide analog is not guanosine or deoxyguanosine. In an embodiment, the nucleotide analog is not a nucleic acid nucleotide. In an embodiment, the nucleotide is not adenosine, guanosine, cytidine, thymidine, uridine, deoxyadenosine, deoxyguanosine, deoxycytidine, deoxythymidine or deoxyuridine.

In one embodiment, the AON of the present invention may comprise one or more nucleotides carrying a 2′-O-(2-methoxy)ethyl (2′-MOE) ribose modification. Also, in one embodiment, the AON comprises one or more nucleotides not carrying a 2′-MOE ribose modification, and wherein the 2′-MOE ribose modifications are at positions that do not prevent the enzyme with adenosine deaminase activity from deaminating the target adenosine. In another embodiment, the AON comprises 2′-O-methyl (2′-O-Me) ribose modifications at the positions that do not comprise a 2′-MOE ribose modification, and/or wherein the oligonucleotide comprises deoxynucleotides at positions that do not comprise a 2′-MOE ribose modification. In one embodiment the AON comprises one or more nucleotides comprising a 2′ position comprising a 2′-MOE, 2′-O-Me, 2′-OH, 2′-deoxy, 2′-fluoro (2′-F), 2′,-2′-difluoro (2′-2′-diF) modification, or a 2′-4′-linkage (i.e., a bridged nucleic acid such as a locked nucleic acid (LNA)). In another embodiment, other nucleic acid monomer that are applied are arabinonucleic acids and 2′-deoxy-2′-fluoroarabinonucleic acid (FANA), for instance for improved affinity purposes. The 2′-4′ linkage can be selected from linkers known in the art, such as a methylene linker or constrained ethyl linker. A wide variety of 2′ modifications are known in the art. Further examples are disclosed in further detail in WO2016/097212, WO2017/220751, WO2018/041973, WO2018/134301, WO2019/219581, and WO2019/158475 for instance. In all cases, the modifications should be compatible with editing such that the oligonucleotide fulfils its role as an editing oligonucleotide, and according to the present invention the modification at the 2′ position of the ribose in the nucleotide analog that is opposite the 5′-G should not interfere with bringing the 5′-G in a syn conformation as outlined herein, or interfere with RNA editing abilities and adenosine deaminase recruitment of the AON when it is attached to its target RNA molecule. Where a monomer comprises an unlocked nucleic acid (UNA) ribose modification, that monomer can have a 2′ position comprising the same modifications discussed above, such as 2′-MOE, 2′-O-Me, 2′-OH, 2′-deoxy, 2′-F, 2′,2′-diF, arabinonucleic acid, FANA, or a 2′-4′-linkage (i.e., a bridged nucleic acids such as a locked nucleic acid (LNA)). Again, in all cases, the modifications should be compatible with editing such that the oligonucleotide fulfils its role as an AON that can, when attached to its target sequence recruit an adenosine deaminase enzyme. In all aspects of the invention, the enzyme with adenosine deaminase activity is preferably ADAR1, ADAR2, or ADAT. In a highly preferred embodiment, the AON is an RNA editing oligonucleotide that targets a pre-mRNA or an mRNA, wherein the target nucleotide is an adenosine in the target RNA, wherein the adenosine is deaminated to an inosine, which is being read as a guanosine by the translation machinery, and wherein the nucleotide that is directly 5′ from the target adenosine is a guanosine. In a further preferred embodiment, the adenosine is in a UGA stop codon, which is edited to a UGG codon. The invention also relates to a pharmaceutical composition comprising the AON as characterized herein, and a pharmaceutically acceptable carrier.

The term ‘nucleoside’ refers to the nucleobase linked to the (deoxy)ribosyl sugar, without phosphate groups. A ‘nucleotide’ is composed of a nucleoside and one or more phosphate groups. The term ‘nucleotide’ thus refers to the respective nucleobase-(deoxy)ribosyl-phospholinker, as well as any chemical modifications of the ribose moiety or the phospho group. Thus, the term would include a nucleotide including a locked ribosyl moiety (comprising a 2′-4′ bridge, comprising a methylene group or any other group), an unlocked nucleic acid (UNA), a nucleotide including a linker comprising a phosphodiester, phosphonoacetate, phosphotriester, PS, phosphoro(di)thioate, MP, phosphoramidate, phosphoryl guanidine linkers, and the like. Sometimes the terms adenosine and adenine, guanosine and guanine, cytidine and cytosine, uracil and uridine, thymine and thymidine/uridine, inosine, and hypoxanthine, are used interchangeably to refer to the corresponding nucleobase on the one hand, and the nucleoside or nucleotide on the other. Sometimes the terms nucleobase, nucleoside and nucleotide are used interchangeably, unless the context clearly requires differently, for instance when a nucleoside is linked to a neighbouring nucleoside and the linkage between these nucleosides is modified. As stated above, a nucleotide is a nucleoside+one or more phosphate groups. The terms ‘ribonucleoside’ and ‘deoxyribonucleoside’, or ‘ribose’ and ‘deoxyribose’ are as used in the art. Whenever reference is made to an oligonucleotide, oligo, ON, ASO, oligonucleotide composition, antisense oligonucleotide, AON, (RNA) editing oligonucleotide, EON, and RNA (antisense) oligonucleotide both oligoribonucleotides and deoxyoligoribonucleotides are meant unless the context dictates otherwise. Whenever reference is made to an ‘oligoribonucleotide’ it may comprise the bases A, G, C, U or I. Whenever reference is made to a ‘deoxyoligoribonucleotide’ it may comprise the bases A, G, C, T or I. However, an AON of the present invention may comprise, besides the nucleoside analog opposite the 5′-G, may comprise a mix of ribonucleosides and deoxyribonucleosides. When a deoxyribonucleoside is used, hence without a modification at the 2′ position of the sugar, the nucleotide is often abbreviated to dA. dC, dG or T in which the ‘d’ represents the deoxy nature of the nucleoside, while a ribonucleoside that is either normal RNA or modified at the 2′ position is often abbreviated without the ‘d’, and often abbreviated with their respective modifications and as explained herein.

Whenever reference is made to nucleotides in the oligonucleotide construct, such as cytosine, 5-methylcytosine, 5-hydroxymethylcytosine, 5-formylcytosine, 5-acetylcytosine, 5-hydroxycytosine, and β-D-glucosyl-5-hydroxymethylcytosine are included; when reference is made to adenine, N6-methyladenine and 7-methyladenine are included; when reference is made to uracil, dihydrouracil, isouracil, N3-glycosylated uracil, pseudouracil, 5-methyluracil, N1-methylpseudouracil, 4-thiouracil and 5-hydroxymethyluracil are included; when reference is made to guanine, 1-methylguanine, 7-methylguanosine, N2,N2-dimethylguanosine, N2,N2,7-trimethylguanosine and N2,7-dimethylguanosine are included. Whenever reference is made to nucleosides or nucleotides, ribofuranose derivatives, such as 2′-deoxy, 2′-hydroxy, and 2′-O-substituted variants, such as 2′-O-methyl, are included, as well as other modifications, including 2′-4′ bridged variants. Whenever reference is made to oligonucleotides, linkages between two mononucleotides may be phosphodiester linkages as well as modifications thereof, including, phosphonoacetate, phosphodiester, phosphotriester, PS, phosphoro(di)thioate, MP, phosphoramidate linkages, thiophosphoramidate, phosphoryl guanidine, thiophosphoryl guanidine, sulfono phosphoramidate and the like.

The term ‘comprising’ encompasses ‘including’ as well as ‘consisting of’, e.g., a composition ‘comprising X’ may consist exclusively of X or may include something additional, e.g., X+Y. The term ‘about’ in relation to a numerical value x is optional and means, e.g., x±10%. The word ‘substantially’ does not exclude ‘completely’, e.g., a composition which is ‘substantially free from Y′ may be completely free from Y. Where relevant, the word ‘substantially’ may be omitted from the definition of the invention.

The term “complementary” as used herein refers to the fact that the AON hybridizes under physiological conditions to the target sequence. The term does not mean that each nucleotide in the AON has a perfect pairing with its opposite nucleotide in the target sequence. In other words, while an AON may be complementary to a target sequence, there may be mismatches, wobbles and/or bulges between AON and the target sequence, while under physiological conditions that AON still hybridizes to the target sequence such that the cellular RNA editing enzymes can edit the target adenosine. The term “substantially complementary” therefore also means that in spite of the presence of the mismatches, wobbles, and/or bulges, the AON has enough matching nucleotides between AON and target sequence that under physiological conditions the AON hybridizes to the target RNA. As shown herein, an AON may be complementary, but may also comprise one or more mismatches, wobbles and/or bulges with the target sequence, if under physiological conditions the AON is able to hybridize to its target.

The term ‘downstream’ in relation to a nucleic acid sequence means further along the sequence in the 3′ direction; the term ‘upstream’ means the converse. Thus, in any sequence encoding a polypeptide, the start codon is upstream of the stop codon in the sense strand but is downstream of the stop codon in the antisense strand.

References to ‘hybridisation’ typically refer to specific hybridisation and exclude non-specific hybridisation. Specific hybridisation can occur under experimental conditions chosen, using techniques well known in the art, to ensure that the majority of stable interactions between probe and target are where the probe and target have at least 70%, preferably at least 80%, more preferably at least 90% sequence identity. The term ‘mismatch’ is used herein to refer to opposing nucleotides in a double stranded RNA complex which do not form perfect base pairs according to the Watson-Crick base pairing rules. In the historical sense, mismatched nucleotides are G-A, C-A, U-C, A-A, G-G, C-C, U-U pairs. In some embodiments AONs of the present invention comprise fewer than four mismatches, for example 0, 1 or 2 mismatches. Wobble base pairs are G-U, I-U, I-A, and I-C base pairs. According to the present invention the G:G pairing and the G:analog pairing would be considered a mismatch, although that does not necessarily mean that the interaction is unstable, which means that the term ‘mismatch’ may be somewhat outdated based on the current invention where a Hoogsteen base-pairing may be seen as a mismatch based on the origin of the nucleotide (analog) but still be relatively stable. An isolated G:G pairing in duplex RNA can for instance be quite stable, but still be defined as a mismatch.

The term ‘splice mutation’ relates to a mutation in a gene that encodes for a pre-mRNA, wherein the splicing machinery is dysfunctional in the sense that splicing of introns from exons is disturbed and due to the aberrant splicing, the subsequent translation is out of frame resulting in premature termination of the encoded protein. Often such shortened proteins are degraded rapidly and do not have any functional activity, as discussed herein. The exact mutation does not have to be the target for the RNA editing; it may be that a neighbouring or nearby adenosine in the (splice) mutation is the target nucleotide, which conversion to I fixes the splice mutation back to a normal state. The skilled person is aware of methods to determine whether normal splicing is restored, after RNA editing of the adenosine within the splice mutation site or area.

An AON according to the present invention may be chemically modified almost in its entirety, for example by providing nucleotides with a 2′-O-methylated sugar moiety (2′-O-Me), 2′-F, and/or with a 2′-O-(2-methoxy)ethyl sugar moiety (2′-MOE). However, the orphan nucleotide is a cytidine, a cytidine analog, a uridine, a uridine analog/derivative, or a nucleotide comprising a Benner's base (as detailed above) and preferably does not comprise the 2′-O-Me or 2′-MOE modification, and in yet a further embodiment, at least one and in another embodiment both the two neighbouring nucleotides flanking each nucleotide opposing the target adenosine further do not comprise a 2′-O-Me modification. Complete modification wherein all nucleotides of the AON hold a 2′-O-Me modification results in a non-functional oligonucleotide as far as RNA editing goes (known in the art), presumably because it hinders the ADAR activity at the targeted position. In general, an adenosine in a target RNA can be protected from editing by providing an opposing nucleotide with a 2′-O-Me group, or by providing a guanine or adenine as opposing base, as these two nucleobases are also able to reduce editing of the opposing adenosine. Various chemistries and modification are known in the field of oligonucleotides that can be readily used in accordance with the invention. The regular internucleosidic linkages between the nucleotides may be altered by mono- or di-thioation of the phosphodiester bonds to yield phosphorothioate esters or phosphorodithioate esters, respectively. Other modifications of the internucleosidic linkages are possible, including amidation and peptide linkers. In an embodiment, the AON of the present invention comprises 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides.

It is known in the art that RNA editing entities (such as human ADAR enzymes) edit dsRNA structures with varying specificity, depending on several factors. One important factor is the degree of complementarity of the two strands making up the dsRNA sequence. Perfect complementarity of the two strands usually causes the catalytic domain of human ADAR to deaminate adenosines in a non-discriminative manner, reacting with any adenosine it encounters. The specificity of hADAR1 and 2 can be increased by introducing chemical modifications and/or ensuring several mismatches in the dsRNA, which presumably help to position the dsRNA binding domains in a way that has not been clearly defined yet. Additionally, the deamination reaction itself can be enhanced by providing an AON that comprises a mismatch opposite the adenosine to be edited. A mismatch as disclosed herein is created by providing a targeting portion having a nucleoside analog opposite the 5′-G from the target adenosine. Following the instructions in the present application, those of skill in the art will be capable of designing the complementary portion of the oligonucleotide according to their needs.

The RNA editing protein present in the cell that is of most interest to be used with AONs of the present invention is human ADAR2. It will be understood by a person having ordinary skill in the art that the extent to which the editing entities inside the cell are redirected to other target sites may be regulated by varying the affinity of the AONs according to the invention for the recognition domain of the editing molecule. The exact modification may be determined through some trial and error and/or through computational methods based on structural interactions between the AON and the recognition domain of the editing molecule. In addition, or alternatively, the degree of recruiting and redirecting the editing entity resident in the cell may be regulated by the dosing and the dosing regimen of the AON. This is something to be determined by the experimenter (in vitro) or the clinician, usually in phase I and/or II clinical trials.

The invention concerns the modification of target RNA sequences in eukaryotic, preferably metazoan, more preferably mammalian, most preferably human cells. The invention can be used with cells from any organ e.g., skin, lung, heart, kidney, liver, pancreas, gut, muscle, gland, eye, brain, blood, and the like. The invention is particularly suitable for modifying sequences in cells, tissues or organs implicated in a diseased state of a (human) subject. The cell can be located in vitro, ex vivo or in vivo. One advantage of the invention is that it can be used with cells in situ in a living organism, but it can also be used with cells in culture. In some embodiments cells are treated ex vivo and are then introduced into a living organism (e.g., re-introduced into an organism from whom they were originally derived). The invention can also be used to edit target RNA sequences in cells from a transplant or within a so-called organoid. Organoids can be thought of as three-dimensional in vitro-derived tissues but are driven using specific conditions to generate individual, isolated tissues. In a therapeutic setting they are useful because they can be derived in vitro from a patient's cells, and the organoids can then be re-introduced to the patient as autologous material which is less likely to be rejected than a normal transplant. The cell to be treated will generally have a genetic mutation. The mutation may be heterozygous or homozygous. The invention will typically be used to modify point mutations, such as N to A mutations, wherein N may be G, C, U (on the DNA level T), preferably G to A mutations, or N to C mutations, wherein N may be A, G, U (on the DNA level T), preferably U to C mutations.

Without wishing to be bound by theory, the RNA editing through hADAR2 is thought to take place on primary transcripts in the nucleus, during transcription or splicing, or in the cytoplasm, where e.g. mature mRNA, miRNA or ncRNA can be edited.

Many genetic diseases are caused by G to A mutations, and these are preferred target diseases because adenosine deamination at the mutated target adenosine will reverse the mutation to a codon giving rise to a functional, full length wild type protein, especially when it concerns PTCs. Especially mutations wherein the target adenosine has a 5′-G in the target sequence can be targeted by the AONs of the present invention.

It should be clear, that targeted editing according to the invention can be applied to any adenosine, whether it is a mutated or a wild-type nucleotide. For example, editing may be used to create RNA sequences with different properties. Such properties may be coding properties (creating proteins with different sequences or length, leading to altered protein properties or functions), or binding properties (causing inhibition or over-expression of the RNA itself or a target or binding partner; entire expression pathways may be altered by recoding miRNAs or their cognate sequences on target RNAs). Protein function or localization may be changed at will, by functional domains or recognition motifs, including but not limited to signal sequences, targeting or localization signals, recognition sites for proteolytic cleavage or co- or post-translational modification, catalytic sites of enzymes, binding sites for binding partners, signals for degradation or activation and so on. These and other forms of RNA and protein “engineering”, whether or not to prevent, delay or treat disease or for any other purpose, in medicine or biotechnology, as diagnostic, prophylactic, therapeutic, research tool or otherwise, are encompassed by the present invention.

The amount of AON to be administered, the dosage and the dosing regimen can vary from cell type to cell type, the disease to be treated, the target population, the mode of administration (e.g., systemic versus local), the severity of disease and the acceptable level of side activity, but these can and should be assessed by trial and error during in vitro research, in pre-clinical and clinical trials. The trials are particularly straightforward when the modified sequence leads to an easily detected phenotypic change. It is possible that higher doses of AON could compete for binding to an ADAR within a cell, thereby depleting the amount of the entity, which is free to take part in RNA editing, but routine dosing trials will reveal any such effects for a given AON and a given target.

One suitable trial technique involves delivering the AON to cell lines, or a test organism and then taking biopsy samples at various time points thereafter. The sequence of the target RNA can be assessed in the biopsy sample and the proportion of cells having the modification can easily be followed. After this trial has been performed once then the knowledge can be retained, and future delivery can be performed without needing to take biopsy samples. A method of the invention can thus include a step of identifying the presence of the desired change in the cell's target RNA sequence, thereby verifying that the target RNA sequence has been modified. This step will typically involve sequencing of the relevant part of the target RNA, or a cDNA copy thereof (or a cDNA copy of a splicing product thereof, in case the target RNA is a pre-mRNA), as discussed above, and the sequence change can thus be easily verified. Alternatively, the change may be assessed on the level of the protein (length, glycosylation, function, or the like), or by some functional read-out, such as a(n) (inducible) current, when the protein encoded by the target RNA sequence is an ion channel, for example.

After RNA editing has occurred in a cell, the modified RNA can become diluted over time, for example due to cell division, limited half-life of the edited RNAs, etc. Thus, in practical therapeutic terms a method of the invention may involve repeated delivery of an AON until enough target RNAs have been modified to provide a tangible benefit to the patient and/or to maintain the benefits over time.

AONs of the invention are particularly suitable for therapeutic use, and so the invention provides a pharmaceutical composition comprising an AON of the invention and a pharmaceutically acceptable carrier. In some embodiments of the invention the pharmaceutically acceptable carrier can simply be a saline solution. This can usefully be isotonic or hypotonic, particularly for pulmonary delivery. The invention also provides a delivery device (e.g., syringe, inhaler, nebuliser) which includes a pharmaceutical composition of the invention.

The invention also provides an AON of the invention for use in a method for making a change in a target RNA sequence in a mammalian, preferably a human cell, as described herein. Similarly, the invention provides the use of an AON of the invention in the manufacture of a medicament for making a change in a target RNA sequence in a mammalian, preferably a human cell, as described herein.

The invention also relates to a method for the deamination of at least one specific target adenosine present in a target RNA sequence in a cell, the method comprising the steps of: providing the cell with an AON according to the invention; allowing uptake by the cell of the AON; allowing annealing of the AON to the target RNA molecule; allowing a mammalian ADAR enzyme comprising a natural dsRNA binding domain as found in the wild type enzyme to deaminate the target adenosine in the target RNA molecule to an inosine; and optionally identifying the presence of the inosine in the RNA sequence.

In a preferred aspect, depending on the ultimate deamination effect of A to I conversion, the identification step comprises: sequencing the target RNA; assessing the presence of a functional, elongated, full length and/or wild type protein; assessing whether splicing of the pre-mRNA was altered by the deamination; or using a functional read-out, wherein the target RNA after the deamination encodes a functional, full length, elongated and/or wild type protein. Because the deamination of the adenosine to an inosine may result in a protein that is no longer suffering from the mutated A at the target position, the identification of the deamination into inosine may also be a functional read-out, for instance an assessment on whether a functional protein is present, or even the assessment that a disease that is caused by the presence of the adenosine is (partly) reversed. The functional assessment for each of the diseases mentioned herein will generally be according to methods known to the skilled person. A very suitable manner to identify the presence of an inosine after deamination of the target adenosine is of course RT-PCR and sequencing, using methods that are well-known to the person skilled in the art.

The AON according to the invention is suitably administrated in aqueous solution, e.g. saline, or in suspension, optionally comprising additives, excipients and other ingredients, compatible with pharmaceutical use, at concentrations ranging from 1 ng/ml to 1 g/ml, preferably from 10 ng/ml to 500 mg/ml, more preferably from 100 ng/ml to 100 mg/ml. Dosage may suitably range from between about 1 μg/kg to about 100 mg/kg, preferably from about 10 μg/kg to about 10 mg/kg, more preferably from about 100 μg/kg to about 1 mg/kg. Administration may be by inhalation (e.g., through nebulization), intranasally, orally, by injection or infusion, intravenously, subcutaneously, intra-dermally, intra-cranially, intravitreally, intramuscularly, intra-tracheally, intra-peritoneally, intra-rectally, parenterally, and the like. Administration may be in solid form, in the form of a powder, a pill, a gel, an eye-drop, a solution, a slow-release formulation, or in any other form compatible with pharmaceutical use in humans.

The contents of all cited references (including literature references, patents, patent applications, and websites) that may be cited throughout this application are hereby expressly incorporated by reference in their entirety for any purpose, as are the references cited therein, in the versions publicly available on Jan. 25, 2023. Protein and nucleic acid sequences identified by database accession number and other information contained in the subject database entries (e.g., non-sequence related content in database entries corresponding to specific Genbank accession numbers) are incorporated by reference, and correspond to the corresponding database release publicly available on Jan. 25, 2023.

### EXAMPLES

### Example 1: General Biochemical Procedures

Bovine serum albumin (BSA) and RNase inhibitor were purchased from New England BioLabs. SDS-polyacrylamide gels were visualized with a Molecular Dynamics 9400 Typhon phosphorimager. Data were analyzed with Molecular Dynamics ImageQuant 5.2 software. All MALDI analyses were performed using a Bruker UltraFlextreme MALDI TOF/TOF mass spectrometer. Oligonucleotide masses were determined with Mongo Oligo Calculator v2.08. Oligonucleotides for sequencing and PCR were purchased from Integrated DNA Technologies or Dharmacon. All other oligonucleotides were synthesized as described below.

### Synthesis of Oligonucleotides

Chemical synthesis for all oligonucleotides was performed using an ABI 394 synthesizer. All protected phosphoramidites were purchased from Glen Research except the 8-azanebularine (azaN) phosphoramidite which was purchased from Berry & Associates. Nucleosides were incorporated during the appropriate cycle on a 0.2 or 1.0 μmol scale. Upon completion of the synthesis, columns were evaporated under reduced pressure for 4h. All oligonucleotides were cleaved from the solid support by treatment with 1:3 ethanol/30% NH4OH at 55° C. for 12 h. The supernatant was transferred to a new screw-cap tube and evaporated under reduced pressure. For all oligonucleotides except the azaN-modified strand, desilylation was performed by resuspending the pellets in anhydrous DMSO and treating TBAF-THF at RT O/N. For the azaN strand, desilyation was carried out in TEA3HF as previously described (Haudenschild B.L. et al. J. Am. Chem. Soc., 2004. 126:11213-11219). To each reaction was added 75 mM sodium acetate in butanol. The oligonucleotides were then precipitated from a solution of 65% butanol at −70° C. for 2 h. The solution was centrifuged at 13,000 rpm for 20 min, supernatant was removed, and the pellet was washed twice with cold 95% ethanol. The RNA pellets were then desalted using a Sephadex G-25 column and purified. Single-stranded RNA oligonucleotides were purified by denaturing polyacrylamide gel electrophoresis and visualized by UV shadowing. Bands were excised from the gel, crushed, and soaked O/N at 4° C. in 0.5 M NaOAc, 0.1% sodium dodecyl sulfate (SDS), and 0.1 mM EDTA. Polyacrylamide fragments were removed with a 0.2 μm filter, and the RNAs were precipitated from a solution of 75% EtOH at −70° C. for 4 h. The solution was centrifuged 13,000 rpm for 20 min and supernatant was removed. The RNA solutions were lyophilized to dryness, resuspended in nuclease-free water, and quantified by absorbance at 260 nm. Oligonucleotide mass was confirmed by MALDI-TOF.

### Preparation of Duplex Substrates for Crystallography

For crystallography, the unmodified RNA strand was purchased from Horizon Dharmacon and purified as described above. Duplex RNA was hybridized in water in a 1:1 ratio by heating to 95° C. for 5 min and slow cooling to 30° C.

### In Vitro Transcription of Editing Target RNAs

Target RNAs for deamination kinetic analyses were transcribed from DNA templates with the MEGAScript T7 Kit (ThermoFisher). DNA digestion was performed using RQ1 RNase-free DNase (Promega). DNase treated RNA product was purified as described above.

### Preparation of Duplex Substrates for ADAR Deamination Kinetics

Purified guide and transcribed RNA were added in a 10:1 ratio to hybridization buffer (180 nM transcribed RNA target, 1.8 μM guide, 1× TE Buffer, 100 mM NaCl), heated to 95° C. for 5 min, and slowly cooled to RT.

### Expression and Purification of Human ADAR2 Constructs for Deamination Kinetics

Human ADAR2 (hADAR2) was overexpressed in S. cerevisiae and as previously described (Macbeth M.R. and B.L. Bass. Methods Enzymol. 2007. 424:319-331). Purification of hADAR2 was carried out by lysing cells in buffer containing 20 mM Tris-HCl, pH 8.0, 5% glycerol, 1 mM BME, 750 mM NaCl, 35 mM imidazole, and 0.01% Nonidet P-40 using a French press. Cell lysate was clarified by centrifugation (19,000 rpm for 1h). Lysate was passed over a 3 mL Ni-NTA column, which was then washed in three steps with 20 mL lysis buffer, wash I buffer (20 mM Tris-HCl, pH 8.0, 5% glycerol, 1 mM BME, 750 mM NaCl, 35 mM imidazole, 0.01% Nonidet P-40), wash∥ buffer (20 mM Tris-HCl, pH 8.0,5% glycerol, 1 mM BME, 35 mM imidazole, 500 mM NaCl), and eluted with 20 mM Tris-HCl, pH 8.0, 5% glycerol, 1 mM BME, 400 mM imidazole, 100 mM NaCl. Fractions containing the target protein were pooled and concentrated to 30-80 μM for use in biochemical assays. Protein concentrations were determined using BSA standards visualized by SYPRO orange staining of SDS-polyacrylamide gels. Purified hADAR2 WT was stored in 20 mM Tris-HCl pH 8.0, 100 mM NaCl, 20% glycerol and 1 mM BME at −70° C.

Expression and Purification of ADAR1 p110 for Deamination Kinetics

MBP-tagged human ADAR1 p110 construct was cloned into a pSc vector using standard PCR techniques. The generated construct (yeast codon optimized) consisted of an N-terminal MBP-tag, a tobacco etch virus (TEV) protease cleavage site followed by the human ADAR1 p110 gene. S. cerevisiae BCY123 cells were transformed with this plasmid and the fusion protein was overexpressed as described (Malik T.N. et al. Nucleic Acids Res. 2021. 49:4020-4036). Purification was carried out by lysing cells in lysis/binding buffer containing 50 mM Tris-HCl, pH 8.0, 5% glycerol, 5 mM 2-mercaptoethanol, 1000 mM KCl, 0.05% NP-40 and 50 μM ZnCl₂ using a microfluidizer. Cell lysate was clarified by centrifugation (39,000×g for 50 min). Lysate was passed over a 2 mL NEB amylose column (pre-equilibrated with binding buffer), which was then washed in 2 steps with 50 mL binding buffer followed by 100 mL wash buffer (50 mM Tris-HCl, pH 8.0, 5% glycerol, 5 mM 2-mercaptoethanol, 500 mM KCl, 0.01% NP-40 and 50 μM ZnCl₂) and eluted with buffer containing 50 mM Tris-HCl, pH 8.0, 10% glycerol, 5 mM 2-mercaptoethanol, 500 mM KCl, 0.01% NP-40, 50 μM ZnCl₂, and 20 mM maltose. Fractions containing the target protein were pooled and dialyzed against a storage buffer containing 50 mM Tris-HCl, pH 8.0, 400 mM KCl, 0.5 mM EDTA, 0.01% NP-40, 10% glycerol and 1 mM tris(2-carboxyethyl)phosphine. Dialyzed protein was concentrated to 2-50 μM and stored as aliquots at −70° C. until further use in biochemical assays. Protein concentrations were determined using BSA standards visualized by SYPRO orange staining of SDS-polyacrylamide gels.

Deamination Assays with ADAR2 and ADAR1 p110

Deamination assays were performed under single-turnover conditions in 15 mM Tris-HCl pH 7.5 3% glycerol, 60 mM KCl, 1.5 mM EDTA, 0.003% Nonidet P-40, 3 mM MgCl₂, 160 U/mL RNAsin, 1.0 μg/mL yeast tRNA, 10 nM RNA, and 75 nM human ADAR2. Each reaction solution was incubated at 30° C. for 30 min before the addition of enzyme. Reactions were then incubated at 30° C. for varying times prior to quenching with 190 μL 95° C. water and heating at 95° C. for 5 min. Reaction products were used to generate cDNA using RT-PCR (Promega Access RT-PCR System). DNA was purified using a DNA Clean & Concentrator kit (Zymo) and subjected to Sanger sequencing via GeneWiz (Azenta). The sequencing peak heights were quantified in SnapGene (Domatics). Data were fit to the equation [P]t=Pf*[1-e\hat{ }(−kobs*t)] for ADAR2 where [P]t is percent edited at time t, [P]f is the final endpoint of editing, and kobs is the observed rate constant. Because of the slower reactions for ADAR1 p110 and lower reaction end point, data were fit to the equation [P]ₜ=0.4*[1-e\hat{ }(−k_{obs}*t)]. Each experiment was carried out in triplicate where the k_{obs} reported is the average of each replicate±standard deviation (SD). Statistical significance between groups was determined by one-way ANOVA using Prism software (GraphPad). For the ADAR1 p110 enzyme, deamination reactions were performed as above with the following modifications: The final reaction solution for ADAR1 p110 contained 15 mM Tris-HCl, pH 7.0 4% glycerol, 26 mM KCl, 40 mM potassium glutamate, 1.5 mM EDTA, 0.003% Nonidet P-40, 160 U/mL RNAsin, 1.0 μg/mL yeast tRNA, and 10 nM RNA, and 250 nM ADAR1 p110.

Expression and Purification of hADAR2 Double Stranded RNA Binding Domain and Deaminase Domain (hADAR2-R2D) for Crystallography

Protein expression and purification were carried out by modifying a previously reported protocol (Macbeth M.R. et al. RNA 2004. 10:1563-1571). S. cerevisiae BCY123 cells were transformed with a pSc-ADAR construct encoding hADAR2-R2D E488Q (corresponding to residues 214-701). Cells were streaked on yeast minimal media minus uracil (CM-ura) plates. A single colony was used to inoculate a 15 mL CM-ura starter culture. After cultures were shaken at 300 rpm and 30° C. overnight, 10 mL of starter culture was used to inoculate each liter of yeast growth medium. After cells reached an OD 600 of 1.5 (approximately 20-24 hrs) cells were induced with 110 mL of sterile 30% galactose per liter and protein was expressed for 6h. Cells were collected by centrifugation at 5000×g for 10 min and stored at −80° C. Cells were lysed in 750 mM NaCl in buffer A (20 mM Tris-HCl, pH 8.0, 5% glycerol, 35 mM imidazole, 1 mM BME, and 0.01% Triton X-100) with a microfluidizer. Cell lysate was clarified by centrifugation (39,000×g for 25 min). Lysate was passed over a 5 mL Ni-NTA column equilibrated with buffer A with 750 mM NaCl, which was then washed in three steps with 50 mL of lysis buffer, wash I buffer (buffer A+300 mM NaCl), and wash II buffer (buffer A+100 mM NaCl). Protein was eluted with a 35-300 mM imidazole gradient in wash II buffer over 80 min at a flow rate of 1 mL/min. Fractions containing target protein were pooled and further purified on a 2 mL GE Healthcare Lifesciences Hi-Trap Heparin HP column in wash II buffer without BME. The His10 fusion protein was washed with 50 mL of wash II buffer without BME and eluted with a 100-1000 mM NaCl gradient over 60 min at a flow rate of 0.8 mL/min. Fractions containing target protein were pooled and cleaved with an optimized ratio of 1 mg of TEV protease per 1 mg of protein. Cleavage was carried out for 2 h at room temperature without agitation before the product was passed over another Ni-NTA column with a flow rate of 0.5 mL/min. The flow through and wash were collected and passed through another Ni-NTA column to remove remaining uncleaved protein. The flow through and wash were collected, dialyzed against 20 mM Tris, pH 8.0, 200 mM NaCl, 5% glycerol, and 1 mM BME, followed by concentration to just under 1 mL for gel filtration on a GE Healthcare HiLoad 16/600 Superdex 200 PG column. Fractions containing purified protein were pooled and concentrated to 7-9 mg/mL for crystallography trials.

Crystallization of the hADAR2-R2D E488Q-(GG)RNA Complex

Crystals of the hADAR2-R2D E488Q-GL11 (GG) RNA complex were grown at RT by the hanging-drop vapordiffusion method. A solution of 0.5 ul volume containing 5.6 mg/ml protein and 47.5 uM Gli1-GG RNA was mixed with 0.5 ul of 50 mM MOPS pH 7.0, 200 mM NaCl, 17% PEG 4000. Crystals took one and half weeks to grow. A cluster of crystals was broken apart and a single cuboid-shaped crystal approximately 100 μm in size was soaked briefly in a solution of mother liquor plus 30% ethylene glycol before flash cooling in liquid nitrogen. Data were collected via wide-phi slicing with 1.0° oscillations on beamline 12-2 at the Stanford Synchrotron Radiation Lightsource. Crystals display anisotropic X-ray diffraction with some diffraction extending beyond 2.2A resolution. The resolution was isotropically truncated to 2.8A resolution to generate a robust complete data set.

Processing and Refinement of Crystallographic Data Diffraction data for the ADAR2-R2D E488Q GL11 (GG)-bound structure were processed with XDS and scaled with XSCALE. The hADAR2d GLI1-bound crystal structure (PDB: 5ED2) was used as a model for molecular replacement by PHENIX. The structures were refined with PHENIX including NCS and zinc coordination restraints. Ideal zinc-ligand distances were determined with average distances found for similar coordination models in the PDB database, including deposited ADAR2 structures. The asymmetric unit includes two protein molecules complexed with RNA. The whole of the double stranded RNA binding domain (residues 215-318) of monomer A, as well as the C-terminal residues 700 and 701 were disordered and were therefore not included in the model. The first 20 residues (215-233), 5′ binding loop residues 462-475 and C-terminal proline (701) of monomer B were disordered and were therefore not included in the model.

### Example 2: Acceleration of Deamination Rates for Full Length ADAR2 and ADAR1 p110 Through a G-G or G-A Pair Adjacent to an Editing Site

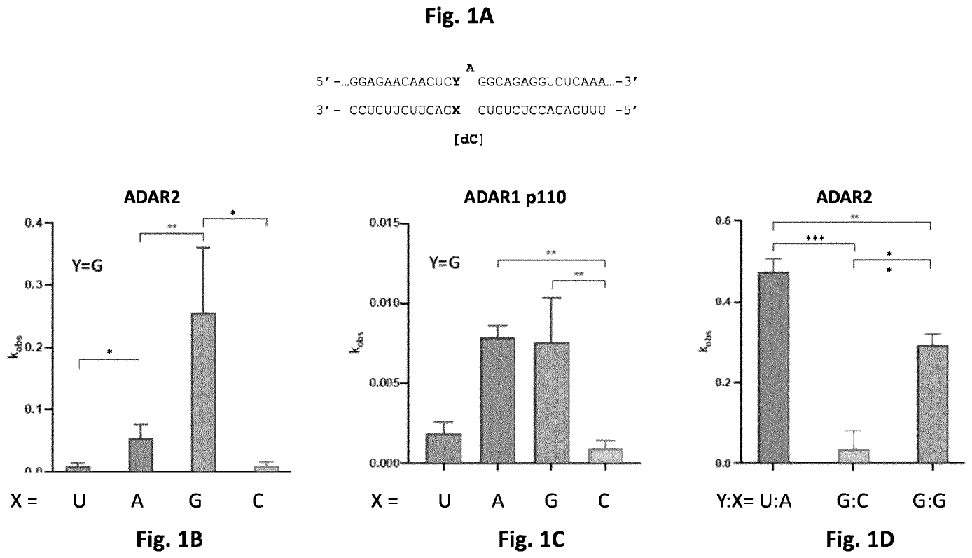

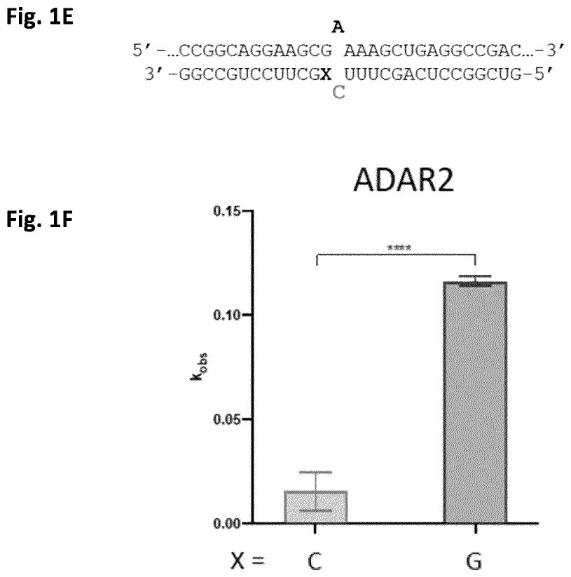

An earlier report describing optimization of guide strands for SNAP-ADARs (in which an ADAR deaminase domain is fused to a SNAP tag and covalently linked to a guiding oligonucleotide) showed that pairing the 5′-G in a 5′-GAG target codon with either A or G in the guide oligonucleotide increased editing efficiency compared to a G:C or G:U pair at that site (Schneider M. F. et al. 2014). Since this location in an RNA substrate is contacted by the ADAR deaminase domain and not by the dsRBDs, it seemed likely that this effect would also be observed with the full length ADARs. However, no such publications appeared available at the time of this invention. WO2021/130313 shows AONs that serve as a guide oligonucleotide targeting the adenosine in the c.5882G>A mutation in human ABCA4, giving rise to Stargardt disease. This mutation has a 5′-G next to the target adenosine and therefore has the disfavored GA configuration. However, the AONs tested therein either comprised a pairing thymidine (dT), a 2-O-methyl modified adenosine (a), or a deoxyadenosine (dA) opposite the 5′-G. Therefore, the inventors of the present invention designed model unmodified RNA substrates for ADAR1 and ADAR2 where the target adenosine is within a 5′-GA-3′ sequence and the 5′-G base pairing partner in the guide oligonucleotide (3′ of the orphan nucleotide) was varied (X═U, A, G, or C), see FIG. 1A. The guide oligonucleotides were also completely RNA except at the indicated positions in the tables and figures. The target adenosine (A) was paired with either 2′-deoxycytidine (FIG. 1A) or cytidine FIG. 1E). The efficiency of each ADAR reaction was then evaluated by measuring deamination rate constants under single turnover conditions (see Table 1 and Table 2). As seen with SNAP-ADARs, the G:G pair and G:A pair led to faster adenosine deamination compared to either G:U or G:C. For the sequence shown in FIG. 1A, ADAR2 deaminated the substrate with the G:G pair at the fastest rate. However, with ADAR1 p110, the substrates with the G:A pair and G:G pair had similar rates, albeit significantly faster than the G:U and G:C substrates. For comparison, the rate of the ADAR2 reaction with a similar substrate RNA bearing a 5′ U paired with A, which is the ideal 5′ nearest neighbor base pair (FIG. 1D) was also measured under conditions where rates for each of these substrates could be measured accurately. While the substrate with the ideal nearest neighbor nucleotides reacts faster, the ADAR2 reaction rate for the 5′ G:G substrate differs by less than two-fold under these conditions (Table 1). Importantly, the rate of deamination of the 5′-G:C substrate was 16-times slower than the ideal substrate under these conditions.

```
TABLE 1
  N RNA
[N] DNA
                                      A
5′-...GGAGAACAACUCY GGCAGAGGUCUCAAA...-3′
3′- CCUCUUGUUGAGX CUGUCUCCAGAGUUU -5′
                                    [C]
```

| enzyme | Y:X | K_{obs} min^{-1d} | Kᵣₑₗ^{e} |
|---|---|---|---|
| ADAR2^{a} | G:G | 0.3 ± 0.1 | 33 |
| | G:C | 0.009 ± 0.006 | 1 |
| | G:A | 0.05 ± 0.02 | 5.5 |
| | G:U | 0.010 ± 0.003 | 1.1 |
| ADAR1 p110^{b} | G:U | 0.0018 ± 0.0007 | 2 |
| | G:A | 0.0076 ± 0.0007 | 8.4 |
| | G:G | 0.008 ± 0.003 | 8.8 |
| | G:C | 0.0009 ± 0.0004 | 1 |
| ADAR2^{c} | G:G | 0.29 ± 0.02 | 10 |
| | G:C | 0.03 ± 0.04 | 1 |
| | U:A | 0.47 ± 0.03 | 16 |

Rate constants for in vitro deamination of a model RNA substrate (from the human IDUA transcript) by ADAR2 and ADAR1 p110. Sequences are identical as shown in FIG. 1A.
ªValues for 100 nM ADAR2 acting on 10 nM RNA substrate.
^{b}Values for 250 nM ADAR1 p110 acting on 10 nM RNA.
^{c}Values for 10 nM ADAR2 acting on 1 nM RNA substrate.
Y:X indicates the base pairing adjacent to the editing site.
^{d}Data for ADAR2 were fitted to the equation [P]t = α[1-exp(−k_{obs}-t)]. Data for ADAR1 p110 were fitted to the equation [P]t = 0.4.[1-exp(-K_{obs}-t)].
^{e}Kᵣₑₗ = K_{obs} for different nucleosides at Y:X position/K_{obs} for Y:X = G:C for each of the conditions a, b, and c.

Notably, the effect of the G:G pair was not limited to a 5‘-GAG’-3′ target sequence. The substrate sequence (from the human MECP2 transcript) shown in FIG. 1E, has the target adenosine within a 5′-GAA-3′ sequence and the ADAR2 reaction with 5′-G:G substrate was nearly eight times faster than the 5′-G:C substrate in this sequence context (Table 2). In conclusion, these results confirm the effect of the G:G and G:A pairs in activating editing at 5′-GA sites for the full length ADARs in vitro.

```
TABLE 2
  N RNA
[N] DNA
                                      A
5′-...CCGGCAGGAAGCG AAAGCUGAGGCCGAC...-3′
3′- GGCCGUCCUUCGX UUUCGACUCCGGCUG -5′
                                      C
```

| enzyme | X | K_{obs} min^{-1a} | kᵣₑₗ^{b} |
|---|---|---|---|
| ADAR2 | G | 0.117 ± 0.002 | 7.8 |
| | C | 0.015 ± 0.009 | 1.0 |

Rate constants for in vitro deamination for 100 nM ADAR2 acting on 10 nM wild type MECP2 substrate. Sequences are identical as shown in FIG. 1E.
X indicates the nucleotide in the −1 position in the AON.
ªData were fitted to the equation [P]t =αa[1-exp(-K_{obs}-t)].
^{b}Kᵣₑₗ = K_{obs} for different nucleosides at X position/K_{obs} for X = C.

### Example 3: ADAR2 Binds a Duplex RNA Substrate with a G_{(Syn)}:G_{(Anti)} Pair Adjacent to the Editing Site

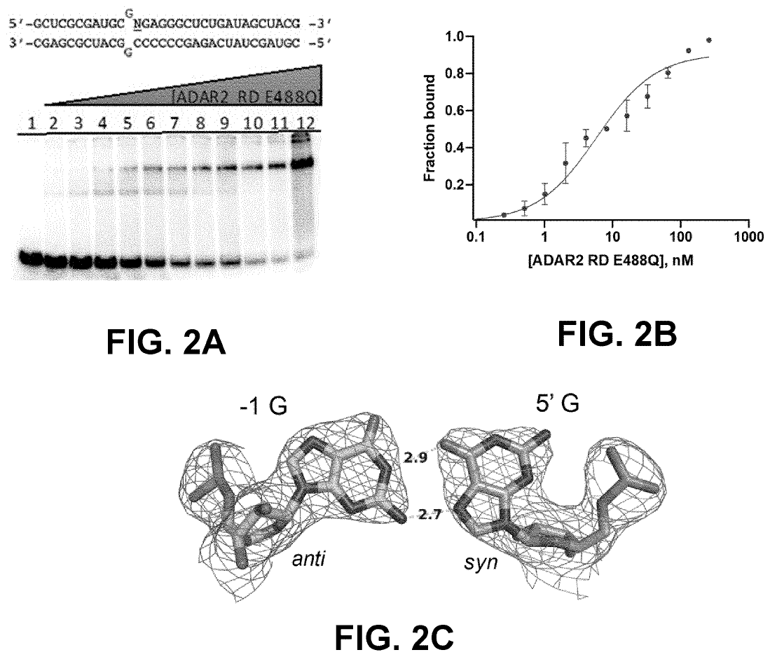

The inventors imagined that a G:G or G:A pair 5′ of the editing site could be superior to a 5′ G:C pair for ADAR deamination of the adjacent adenosine in two possible ways. First, since ADARs must distort the duplex RNA and flip the adenosine out of the double helix, a purine:purine pair adjacent to the editing site might destabilize the duplex and facilitate the needed conformational changes. On the other hand, the G:G and G:A combinations may form stable hydrogen bonded pairs whose minor groove structures are more compatible with ADAR binding than that of a Watson-Crick G:C pair. Indeed, several different H-bonded G:G and G:A pairs have been observed in high resolution structures of RNA (Leontis N. B. and E. Westhof. RNA 2001. 7:499-512). The inventors reasoned that the knowledge of the nature of the purine:purine interaction could potentially provide information about the design and use of nucleoside analogs for enabling ADAR editing within 5′-GA sequences in the target RNA molecule. Therefore, to gain greater insight into the structure of an ADAR-RNA complex with a G:G pair adjacent to an editing site the inventors turned to X-ray crystallography. A 32 bp duplex bearing 8-azanebularine (N) at a known editing site (see Matthews et al. 2016) for trapping an ADAR-bound complex was designed, see FIG. 2A. The use of 8-azanebularine (N) at the editing site allowed a stable ADAR-RNA structure in a catalytically relevant state for structure studies. When N is properly positioned within an ADAR substrate RNA, ADARs promote its covalent hydration to form a structure that mimics the adenosine deamination transition state (Matthews et al. 2016; Haudenschild et al. 2004). A G:G pair adjacent to the N was introduced and its interaction with ADAR2 RD E488Q was evaluated. In earlier work, it was found that this combination of ADAR2 mutant and RNA duplex is conducive to study by X-ray crystallography (Thuy-Boun A.S. et al. Nucleic Acids Res. 2020. 48(14):7958-7972). ADAR2 RD E488Q formed a well-defined complex with this duplex as seen in EMSA gels and bound with K_{d}=6±2 nM (FIG. 2B). It turned out to be possible to form protein/RNA crystals using the 32 bp 8-azanebularine-containing duplex with the G:G pairing and ADAR2-R2D E488Q that diffracted X-rays beyond 2.8 Å resolution. As observed before, the protein bound the RNA as an asymmetric dimer with the deaminase domain of one monomer involved in direct RNA binding to the flipped-out N nucleoside (crystal structure not shown). Importantly, the G:G adjacent to the editing site is well resolved with electron density that best fits a G_{syn}:Gₐₙₜᵢ pair with the guanosine on the 5′ side of the N in a syn conformation with its Hoogsteen face accepting two hydrogen bonds from the Watson-Crick face of the guanosine on the opposing strand (FIG. 2C). The guanosine of this strand is in an anti-conformation. The G:G pairing involves N1 to 06 and 2-amino to N7 hydrogen bonding seen in other G_{syn}:Gₐₙₜᵢ pairs in RNA (Burkhard M. E. and D. H. Turner. Biochemistry 2000. 39:11748-11762; Jiang F. etal. Nature 1996. 382:183-186). This pairing orientation places the 2-amino group of the guanosine on the edited strand in the major groove away from any potential clash with ADAR (FIG. 3A; arrow pointing at the position of the 2-amino group). Indeed, the minor groove edge of the G_{syn}:Gₐₙₜᵢ pair accommodates flipping loop residue G489 in a manner very similar to that of the A:U pair in previously reported ADAR-RNA structures (FIG. 3B) (Matthews et al. 2016).

### Example 4: Use of Nucleoside Analogs Paired with the 5′-G to Further Improve Editing Efficiency

The inventors reasoned that the observation of a now well-defined G_{syn}:Gₐₙₜᵢ pair in the ADAR-RNA complex allowed the use of chemical modifications of the nucleoside opposite the 5′-G, to further modulate the deamination efficiency. To test this, an initial seven 29 nt ADAR guide oligonucleotides were generated that varied at this position to include several different nucleoside analogs, see FIG. 4A in which X═guanosine (G), adenosine (A), 2′-deoxyguanosine (dG), 2′-deoxyadenosine (dA), 7-deaza-2′-deoxyguanosine (7-deaza dG), 8-bromo-2′-deoxyguanosine (8-bromo dG), or 3-deaza-2′-deoxyadenosine (3-deaza dA). The analogs chosen for testing varied in preferred sugar pucker, sterics and hydrogen-bonding capabilities. The guide RNA sequence was designed to recruit ADARs to induce a potentially corrective edit at the premature termination codon generated by the R255X mutation in the MECP2 gene associated with Rett Syndrome. The disease-associated C to T mutation leads to a UGA termination codon in the MECP2 transcript. While ADAR editing is not capable of restoring the wild-type sequence (CGA), it can convert the termination codon to one for tryptophan (UGG) leading to expression of full length R255W (albeit mutant) MECP2 protein. RNA duplexes were formed with the modified guides and an RNA transcript bearing the MECP2 R255X sequence and the rate constants for deamination at the target adenosine by ADAR2 under single turnover conditions was measured (Table 3).

```
TABLE 3
                                      A
5′-...CCGGCAGGAAGUG AAAGCUGAGGCCGAC...-3′
3′- GGCCGUCCUUCAX UUUCGACUCCGGCUG -5′
                                      C
```

| enzyme | X | k_{obs} min^{-1a} | kᵣₑₗ^{b} |
|---|---|---|---|
| ADAR2 | G | 0.73 ± 0.04 | 1 |
| | A | 0.14 ± 0.06 | 0.2 |
| | dG | 0.4 ± 0.1 | 0.5 |
| | dA | 0.4 ± 0.2 | 0.5 |
| | 7-deaza dG | 0.028 ± 0.006 | 0.04 |
| | 8-Br dG | 0.05 ± 0.02 | 0.07 |
| | 3-deaza dA | 1.2 ± 0.2 | 1.6 |

Rate constants for in vitro deamination for 100 nM ADAR acting on 10 nM substrate bearing nucleoside analogs paired with 5′-G (X position). Sequences are identical as shown in FIG. 1E, and FIG. 4A. X = guanosine (G), adenosine (A), 2′-deoxyguanosine (dG), 2′-deoxyadenosine (dA), 7-deaza-2′-deoxyguanosine (7-deaza dG), 8-bromo-2′-deoxyguanosine (8-bromo dG) and 3-deaza-2′-deoxyadenosine (3-deaza dA)
^{a}Data were fitted to the equation [P]t = α[1-exp(-K_{obs}-t)].
^{b}kᵣₑₗ = K_{obs} for different nucleosides at X position/K_{obs} for X = G.

The slow reaction with ADAR1 p110 prevented accurately measuring rates for this substrate in vitro. As seen with the sequences described above, ADAR2 reacts faster with the substrate bearing a G:G pair compared to G:A pair. The 2′-deoxy modification is well tolerated as both dG and dA support reaction with ADAR2. This is consistent with the 2′-endo sugar pucker observed for Gₐₙₜᵢ found in the G:G pair in the structure described above. The biggest differences in reaction rates were observed with the base-modified purines. Both 7-deaza dG and 8-bromo dG led to very slow deamination reactions. This was not unexpected because for instance 8-bromo dG has a relatively “big” atom attached to carbon 8 of the base, which is an unfavored sterics condition. This forces this nucleoside in the AON to adopt a syn conformation. Since syn-syn is expected to be combination that would be unfavorable for interaction with ADAR and provide inefficient editing, this analog was predicted to be inefficient, and it was. On the other hand, 3-deaza dA paired with the 5′-G led to the fastest ADAR2 reaction.

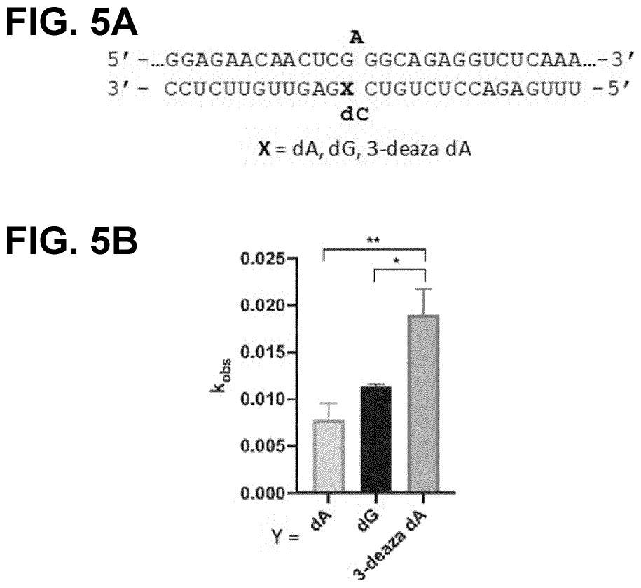

While the MECP2 R255X RNA duplex was a poor substrate for ADAR1 p110 in vitro, the inventors were able to measure rates of deamination for this enzyme using the model substrate shown in FIG. 1A. Therefore, dG, dA and 3-deaza-dA were introduced into the site paired with the 5′-G in that substrate RNA and measured rate constants for deamination by ADAR1 p110 (FIGS. 5A-5B; Table 4). It was found that, like ADAR2, ADAR1 p110 deaminated the substrate bearing the 3-deaza dA paired with the 5′-G at the fastest rate.

```
TABLE 4
  N RNA
[N] DNA
                                      A
5′-...GGAGAACAACUCG GGCAGAGGUCUCAAA...-3′
3′- CCUCUUGUUGAGX CUGUCUCCAGAGUUU -5′
                                    [C]
```

| enzyme | X | K_{obs} min^{-1a} | Kᵣₑₗ^{b} |
|---|---|---|---|
| ADAR1 p110 | G | 0.008 ± 0.003 | 1 |
| | A | 0.0076 ± 0.0007 | 0.9 |
| | dG | 0.0112 ± 0.0005 | 1.4 |
| | dA | 0.007 ± 0.002 | 0.9 |
| | 3-deaza dA | 0.019 ± 0.002 | 2.3 |

Rate constants for in vitro deamination for 250 nM ADAR acting on 10 nM substrate bearing nucleoside analogs paired with 5′-G (X position). Sequences are identical as shown in FIG. 1A. X = guanosine (G), adenosine (A), 2′-deoxyguanosine (dG), 2′-deoxyadenosine (dA) and 3-deaza-2′-deoxyadenosine (3-deaza dA)
^{a}Data were fitted to the equation [P]t = 0.4.[1-exp(-k_{obs}-t)].
^{b}Kᵣₑₗ = K_{obs} for different nucleosides at X position/K_{obs} for X = G.

### Example 5: Use of Nucleoside Analogs Paired with the 5′-G to Further Improve Editing Efficiency

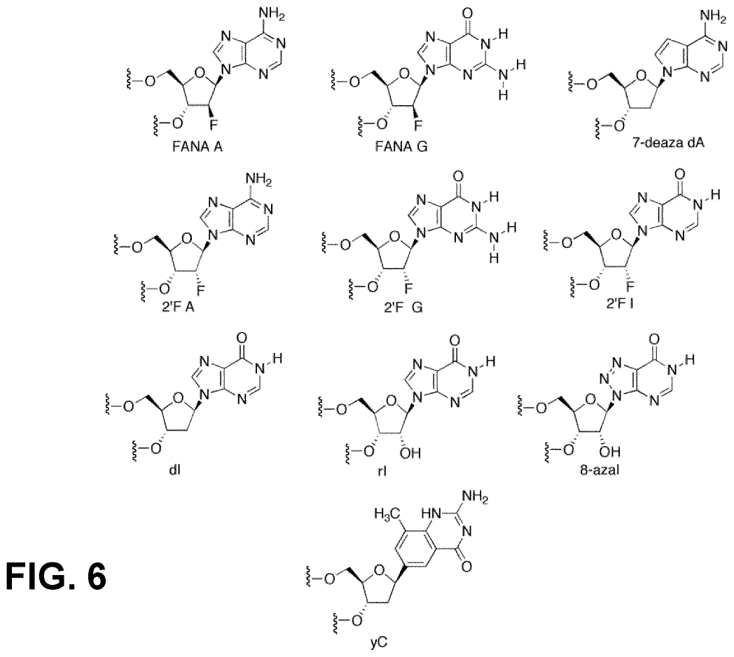

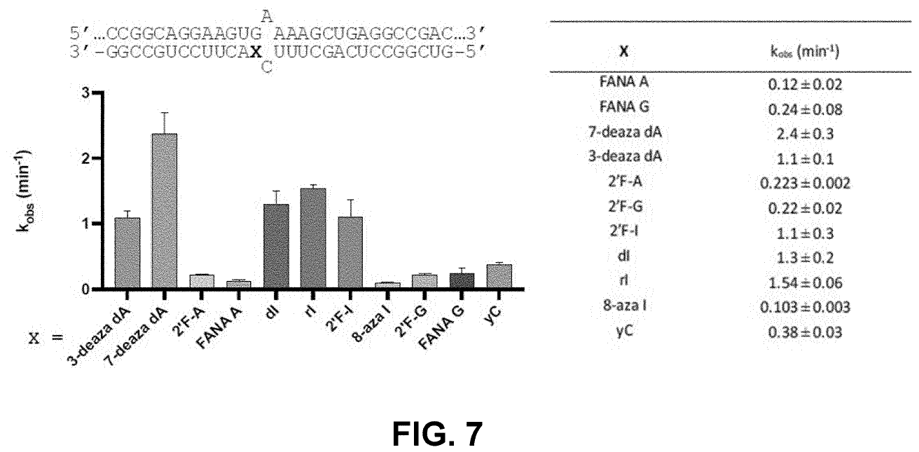

The same experiment as outlined in Example 4 was performed using a further set of ADAR guide oligonucleotides that varied at the X position to include several different nucleoside analogs in which X was one of the nucleotide analogs as shown in FIG. 6. Again, the rate constants for deamination at the target adenosine by ADAR2 under single turnover conditions was measured, see the table in FIG. 7. FIG. 7 also shows the results of the table in a bar diagram, showing the further beneficial properties of the 7-deaza-purine nucleotide analog (7-deaza-2′-deoxyadenosine; 7-deaza dA), in comparison to 3-deaza dA and other nucleotide analogs tested in Example 4.

### CONCLUSIONS

ADARs have a well-established preference for editing adenosines with 5′ nearest neighbor U (or A) and against sites with 5′ nearest neighbor G (Eggington et al. 2011; Li J.B. et al. Science, 2009. 324(5931):1210-1213; Eifler T. et al. Biochemistry, 2013. 52(45):7857-7869). This limits the efficiency of therapeutic directed RNA editing applications where the target adenosine has a 5′-G, such as premature UGA termination codons. Examples are those arising from the R168X, R255X and R270X mutations in the MECP2 gene associated with Rett Syndrome. Earlier, it was found that structures of ADAR2 bound to RNA bearing a 5′ nearest neighbor U revealed a loop of the protein involved in stabilizing the flipped-out conformation (i.e., aa S486-G489) that occupied the RNA minor groove spanning three base pairs that included the nearest neighbor nucleotides and the edited base (Matthews et al. 2016; Thuy-Boun et al. 2020; Monteleone L. R. et al. Cell Chem. Biol. 2019. 26:269-277; Doherty E. E. et al. J. Am. Chem. Soc. 2021. 143:6865-6876). The minor groove edge of the base pair that includes the 5′ nearest neighbor base was juxtaposed to the protein backbone at G489. Modelling a G-C pair at this position (i.e., 5′-G) suggested the guanine 2-amino group in the minor groove would clash with the protein at G489. In an earlier study, it was also shown that replacing a U-A pair at this position with a U-2-aminopurine (2AP) pair resulted in an 80% reduction in deamination rate, further illustrating the detrimental effect of the amino group in the minor groove at this location (Matthews et al. 2016). The G_{syn}:Gₐₙₜᵢ pair solves this apparent steric problem by inducing a change in the glycosidic bond angle at the 5′-G from anti to syn, moving the offending 2-amino group into the major groove where it does not clash with ADAR. This explanation for the effect of the G:G pair seems more likely than the simple effect of a purine:purine mismatch destabilizing the duplex and facilitating base flipping. This is particularly apparent when one considers the fact that the structure of the purine paired with the 5′-G can have a very large effect on the rate enhancement observed. For instance, the substrate with 7-deaza dA paired with a 5′-G reacts with ADAR2 over 85-fold faster than the substrate with 7-deaza dG paired with the 5′-G. Thus, the formation of the G_{syn}:Gₐₙₜᵢ pair with the editing site 5′ nearest neighbor G in the syn conformation explains why the G:G pair on the 5′ side of an editing site enhances ADAR editing compared to G:C or G:U. However, pairing a 5′-G with A also increases the ADAR rate compared to G:C and G:U. Importantly, A is also capable of forming a stable pair with G where the G is in a syn conformation. When protonated at N1, AH+can donate two hydrogen bonds to the Hoogsteen face of G forming the G_{syn}:AH+ₐₙₜᵢ pair (FIG. 4C) (Pan et al. 1999). The increase in deamination rate observed with 7-deaza dA and 3-deaza dA compared to dA paired with the 5′ G is consistent with the formation of the G_{syn}:AH+ₐₙₜᵢ pair. The N1H-N7 hydrogen bond of this pair requires protonation of the adenine ring (FIG. 4C). Since the pKa for N1 protonation for both 3-deazaadenosine (6.8) and 7-deazaadenosine (5.3) are both substantially higher than for adenosine (3.7), this site is more likely to bear a proton available for hydrogen bonding in the deazaadenosine systems under the condition of the ADAR reaction. Interestingly, the opposite effect is observed for 7-deaza dG compared to dG. In this case, the higher N1H pKa of the 7-deazaguanosine (10.3) compared to guanosine (9.5) weakens the N1H-06 hydrogen bond in the G_{syn}:Gₐₙₜᵢ base pair. Indeed, the art showed that 7-deazaguanosine substitution for either guanosine of a G_{syn}:Gₐₙₜᵢ pair is substantially destabilizing in duplex RNA and suggested this was due, at least in part, to the weaker N1H-06 hydrogen bond formed by 7-deazaguanosine (Burkhard and Turner. 2000). Here, it was also shown that 8-bromo dG opposite the 5′ nearest neighbor G led to a slow ADAR2 reaction. This was not surprising given the propensity for this nucleoside to adopt a syn conformation rendering it incompatible with the type of G_{syn}:Gₐₙₜᵢ pair observed in the ADAR complex (Ikehara M. et al. Biochemistry, 1972. 11(5):830-836).

In conclusion, the combination of deamination kinetics and structural studies described here identified an approach to facilitate ADAR editing at challenging 5′-GA sites. The use of nucleosides capable of hydrogen bonding to the Hoogsteen face of the 5′-G and inducing a syn conformation at this location in the RNA without also introducing additional sterically demanding groups into the minor groove enables efficient editing at these sites.

SEQUENCES (all 5′ to 3′)

SEQ ID NO: 1 (RNA)
GGAGAACAACUCYAGGCAGAGGUCUCAAA (Y = U/G)
SEQ ID NO: 2-4 (RNA and/or DNA)
UUUGAGACCUCUGUCCXGAGUUGUUCUCC
(SEQ ID NO 2: X = U, G, C, A)
(SEQ ID NO 3: dA, dG)
(SEQ ID NO 4: 3-deaza-2′-deoxyadenosine (3-deaza dA))
SEQ ID NO: 5 (RNA)
CCGGCAGGAAGCGAAAAGCUGAGGCCGAC
SEQ ID NO: 6 (RNA and/or DNA)
GUCGGCCUCAGCUUUCXGCUUCCUGCCGG
(X = C/G)
SEQ ID NO: 7 (RNA)
GCUCGCGAUGCGNGAGGGCUCUGAUAGCUACG (N = 8-azanebularine)
SEQ ID NO: 8 (RNA and/or DNA)
CGUAGCUAUCAGAGCCCCCCGGCAUCGCGAGC
SEQ ID NO: 9 (RNA)
CCGGCAGGAAGUGAAAAGCUGAGGCCGAC
SEQ ID NO: 10-24 (RNA and/or DNA)
GUCGGCCUCAGCUUUCXGCUUCCUGCCGG
(SEQ ID NO 10: X = G, A)
(SEQ ID NO 11: X = dG, dA)
(SEQ ID NO 12: X = 7-deaza-2′-deoxyguanosine (7-deaza dG))
(SEQ ID NO 13: X = 8-bromo-2′-deoxyguanosine (8-bromo dG))
(SEQ ID NO 14: X = 7-deaza-2′-deoxyadenosine (7-deaza dA))
(SEQ ID NO 15: X = 2′-ara-fluoro adenosine (FANA A))
(SEQ ID NO 16: X = 2′-ara-fluoro guanosine FANA G))
(SEQ ID NO 17: X = 2′-fluoroadenosine (2′F-A))
(SEQ ID NO 18: X = 2′-fluoroguanosine (2′-F-G))
(SEQ ID NO 19: X = 2′-fluoroinosine (2′F-I))
(SEQ ID NO 20: X = 2′-deoxyinosine (dl))
(SEQ ID NO 21: X = 2′-OH-inosine (rl))
(SEQ ID NO 22: X = 8-aza-inosine)
(SEQ ID NO 23: X = 1′-(2-amino-8-methyl-4-oxo-1,4-dihydro-6-quinazolinyl)-2′-deoxyribose
(yC))
(SEQ ID NO 24: X = 3-deaza-2′-deoxyadenosine (3-deaza dA))

1. An antisense oligonucleotide (AON) capable of forming a double stranded nucleic acid complex with a target RNA molecule, wherein the double stranded nucleic acid complex is capable of recruiting an adenosine deaminating enzyme for deamination of a target adenosine in the target RNA molecule, wherein the nucleotide directly 5′ of the target adenosine in the target RNA molecule is a guanosine, and wherein the nucleotide in the AON that is opposite the guanosine is a nucleotide analog that can induce a syn conformation of the guanosine.

2. The AON according to claim 1, wherein at least one nucleotide or nucleotide analog in the AON comprises a substitution at the 2′ position of the ribose, wherein the substitution is selected from the group consisting of:
H (DNA);
OH (RNA);
F;
ara-F
diF;
substituted or unsubstituted, linear or branched lower (C1-C10) alkyl, alkenyl, alkynyl, alkaryl, allyl, or aralkyl, that may be interrupted by one or more heteroatoms;
O-, S-, or N-alkyl;
O-, S-, or N-alkenyl;
O-, S-, or N-alkynyl;
O-, S-, or N-allyl;
O-alkyl-O-alkyl;
methoxy;
aminopropoxy;
methoxyethoxy;
dimethylamino oxyethoxy; and
dimethylaminoethoxyethoxy, with the proviso that the nucleotide that is opposite the target adenosine does not comprise a 2′-O-methyl or a 2′-(2-methoxy)ethyl ribose modification.

3. The AON according to claim 1 or 2, wherein the nucleotide analog comprises a modified nucleobase.

4. The AON according to any one of claims 1 to 3, wherein the nucleotide analog comprises a modified purine nucleobase.

5. The AON according to claim 4, wherein the modified purine nucleobase comprises a 7-deaza-purine modification (such as a 7-deaza-adenine modification), or a 3-deaza-purine modification (such as a 3-deaza-adenine modification).

6. The AON according to claim 4 or 5, wherein the modified purine nucleobase comprises a hydrogen bond donor at N1 that has a pKa that is higher than 3.7 and lower than 9.5.

7. The AON according to any one of claims 1 to 6, wherein the nucleotide analog is selected from the group consisting of:
7-deaza-2′-deoxyadenosine (7-deaza dA);
7-deaza-2′-adenosine (7-deaza A);
7-deaza-2′-deoxy-2′-fluoroadenosine (7-deaza fA);
7-deaza-2′-deoxy-2′-ara-fluoroadenosine;
7-deaza-2′-deoxy-2′,2′-difluoroadenosine;
3-deaza-2′-deoxyadenosine (3-deaza dA);
3-deaza-2′-adenosine (3-deaza A);
3-deaza-2′-deoxy-2′-fluoroadenosine (3-deaza fA);
3-deaza-2′-deoxy-2′-ara-fluoroadenosine;
3-deaza-2′-deoxy-2′,2′-difluoroadenosine;
3,7-dideaza-2′-deoxyadenosine (3,7-dideaza dA);
3,7-dideaza-2′-adenosine (3,7-dideaza A);
3,7-dideaza-2′-deoxy-2′-fluoroadenosine (3,7-dideaza fA);
3,7-dideaza-2′-deoxy-2′-ara-fluoroadenosine;
3,7-dideaza-2′-deoxy-2′,2′-difluoroadenosine;
3-deaza-2′-O-[2-(methoxy)ethyl]adenosine;
3-deaza-2′-O-[2-methylamino-2-oxoethyl]adenosine;
2′-deoxy-2′-fluoroguanosine;
2′-ara-fluoro guanosine (FANA G);
2′,2′-difluoro guanosine;
2′-deoxyinosine (dl);
2′-OH-inosine (rl);
2′-fluoroinosine (2′-F-1);
2′-ara-fluoro inosine (FANA I);
2′,2′-difluoro inosine;
5-formylindole-2′-deoxyriboside;
5-formyl-2′-fluoro-2′-deoxyriboside
5-formylindole-2′-ara-fluoro-2′-deoxyriboside;
5-formylindole-2′,2′-difluoro-2′-deoxyriboside;
5-formylindole-2′-O-methylriboside;
5-formylindole-2′-O-[2-(methoxy)ethyl]riboside;
5-formylindole-2′-O-[2-methylamino-2-oxoethyl]riboside
beta-(4-amidino-1H-imidazol-1-yl) riboside;
beta-(4-amidino-1H-imidazol-1-yl) 2′-deoxyriboside;
beta-(4-amidino-1H-imidazol-1-yl) 2′-ara-fluoro-2′-deoxyriboside; and
beta-(4-amidino-1H-imidazol-1-yl) 2′,2′-difluoro-2′-deoxyriboside.

8. The AON according to any one of claims 1 to 7, wherein the adenosine deaminating enzyme is an endogenous ADAR enzyme.

9. The AON according to any one of claims 1 to 8, wherein the AON comprises at least one phosphorothioate (PS), phosphonoacetate, phosphoramidate, phosphoryl guanidine or a methylphosphonate (MP) internucleotide linkage.

10. The AON according to any one of claims 1 to 9, wherein the AON comprises at least 15, 16, 17, 18, 19, 20, 21, 22, 23, 24, 25, 26, 27, 28, 29, 30, 31, 32, 33, 34, 35, or 36 nucleotides, and is at most 100 nucleotides long.

11. A pharmaceutical composition comprising an AON according to any one of claims 1 to 10, and a pharmaceutically acceptable carrier or diluent.

12. An AON according to any one of claims 1 to 10, or a pharmaceutical composition according to claim 11, for use in the treatment, amelioration, or slowing down progression of a genetic disease caused by a premature UGA termination codon.

13. A method for the deamination of at least one target adenosine present in a target RNA molecule in a cell, wherein the nucleotide 5′ of the target adenosine in the RNA molecule is a guanosine, the method comprising the steps of:
(i) providing the cell with an AON according to any one of claims 1 to 10, or a pharmaceutical composition according to claim 11;
(ii) allowing annealing of the AON to the target RNA molecule to form a double stranded nucleic acid complex capable of recruiting an adenosine deaminating enzyme in the cell, preferably an endogenous adenosine deaminating enzyme;
(iii) allowing the adenosine deaminating enzyme to deaminate the target adenosine in the target RNA molecule; and
(iv) optionally identifying the presence of the deaminated adenosine in the target RNA molecule.

14. The method of claim 13, wherein step (iv) comprises:
a) sequencing a region of the target RNA molecule, wherein the region comprises the deaminated target adenosine;
b) assessing the presence of a functional, elongated, full length and/or wild type protein when the target adenosine is in a UGA stop codon; or
c) using a functional read-out, wherein the target RNA molecule after the deamination encodes a functional, full length, elongated and/or wild type protein.

15. A method for the deamination of at least one target adenosine present in a target RNA molecule, the method comprising the steps of:
(i) providing an AON according to any one of claims 1 to 10, or a pharmaceutical composition according to claim 11;
(ii) allowing annealing of the AON to the target RNA molecule to form a double stranded nucleic acid complex with the target RNA molecule;
(iii) allowing a mammalian adenosine deaminating enzyme to deaminate the target adenosine in the target RNA molecule; and
(iv) optionally identifying the presence of the deaminated adenosine in the target RNA molecule.

16. A method according to any one of claims 13 to 15, wherein the adenosine deaminating enzyme is an endogenous ADAR enzyme.

17. An AON according to any one of claims 1 to 10, or a pharmaceutical composition according to claim 11, for use in the treatment of a genetic disorder, preferably selected from the group consisting of: Hurler Syndrome, alpha-1-antitrypsin (A1AT) deficiency, (familial) hypercholesterolemia, Parkinson's disease, Rett syndrome, Stargardt Disease, Citrullinemia Type 1, autosomal recessive non-syndromic hearing loss, X-linked retinoschisis, argininosuccinate lyase deficiency, Duchenne/Becker muscular dystrophy, Non-Alcoholic Steatohepatitis (NASH), Myotonic dystrophy type I, Myotonic dystrophy type II, Huntington's disease, Usher syndrome (such as Usher syndrome type I, II, and Ill), Charcot-Marie-Tooth disease, Cystic fibrosis, Alzheimer's disease, albinism, Amyotrophic lateral sclerosis, Asthma, B-thalassemia, Epileptic Encephalopathy, CADASIL syndrome, Chronic Obstructive Pulmonary Disease (COPD), Distal Spinal Muscular Atrophy (DSMA), Dystrophic Epidermolysis bullosa, Epidermolysis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hereditary Cancer predisposing Syndrome, Hunter Syndrome, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, neurofibromatosis, Niemann-Pick disease type A, B and C, NY-eso1 related cancer, Peutz-Jeghers Syndrome, Phenylketonuria, Pompe's disease, Primary Ciliary Disease, Prothrombin mutation related disorders, such as the Prothrombin G20210 Å mutation, Pulmonary Hypertension, (autosomal dominant) Retinitis Pigmentosa, Sandhoff Disease, Severe Combined Immune Deficiency Syndrome (SCID), Sickle Cell Anaemia, Spinal Muscular Atrophy, Tay-Sachs Disease, X-linked immunodeficiency, Sturge-Weber Syndrome, and cancer, such as breast and lung cancer.
